(19) United States
(12) Patent Application Publication
(10) Pub. No.: US 2022/0259309 A1
(43) Pub. Date: Aug. 18, 2022

(54) ANTIBODY COMBINATIONS FOR TREATMENT OF CANCER IN SPECIFIC PATIENTS

(71) Applicants: BIOINVENT INTERNATIONAL AB, Lund (SE); UNIVERSITY OF SOUTHAMPTON, Southampton Hampshire (GB)

(72) Inventors: Björn FRENDÉUS, Lund (SE); Ingrid TEIGE, Lund (SE); Linda MÅRTENSSON, Bjärred (SE); Ingrid KARLSSON, Lund (SE); Mark CRAGG, Southampton Hampshire (GB); Stephen BEERS, Southampton Hampshire (GB); Robert OLDHAM, Southampton Hampshire (GB)

(21) Appl. No.: 17/627,385

(22) PCT Filed: Jul. 17, 2020
(86) PCT No.: PCT/EP2020/070319

§ 371 Date: Jan. 14, 2022

(30) Foreign Application Priority Data
Jul. 17, 2019 (EP) 19186840.5

(51) Int. Cl. C07K 16/28; A61P 35/00

(52) U.S. Cl. CPC C07K 16/283; A61K 2039/507; A61P 35/00; C07K 16/2818

(57) ABSTRACT

Described is the combined use of a first antibody molecule that specifically binds FcyRIIb via its Fab region and that binds an Fey receptor via its Fc region, and a second antibody molecule that specifically binds PD-1 and that binds at least one Fey receptor via its Fc region, in the treatment of cancer in a patient having tumor infiltrating T lymphocytes with a medium or high PD-1 expression, as well as pharmaceutical compositions and kits comprising these two antibody molecules, and methods of treating cancer using these two antibodies. Described is also a diagnostic test for identification of patients benefitting from the treatment described herein.

### FIELD OF THE INVENTION

The present invention relates to the combined use of 1) a first antibody molecule that specifically binds FcγRIIb via its Fab region, and that binds an Fcγ receptor via its Fc region, and 2) a second antibody molecule that specifically binds to PD-1 and that binds to at least one Fcγ receptor via its Fc region in the treatment of cancer in a patient who has medium or high expression of PD-1 on the CD3 positive tumor-infiltrating lymphocytes (TILs).

### BACKGROUND OF THE INVENTION

Immune inhibitory checkpoint receptors, e.g. CTLA-4 or PD-1 (also denoted PD1), are cell surface receptors that upon binding of their ligand receptors, e.g. B7 family members CD80 and CD86, and PD-L1, respectively, transmit inhibitory signals into the cell interior, limiting cell activation and proliferation, preventing excessive inflammation and contributing to maintenance of self-tolerance. Animals genetically deficient in such inhibitory immune checkpoints are associated with exacerbated inflammatory responses, failing to develop or maintain tolerance to self, resulting in autoimmune disease. Antibodies to immune checkpoint receptors CTLA-4 and PD-1/PD-L1 elicit increased overall survival of patients with various cancers, notably including multiple solid cancer types, e.g. melanoma, lung, bladder, and head and neck cancer, and such antibodies have been approved by the U.S. Food and Drug Administration (Pardoll, D. M. (2012) Nat Rev Cancer 12(4): 252-264; Topalian, S. L. et al (2015) Cancer Cell 27(4): 450-461; Sharma, P. et al (2017) Cell 168(4): 707-723).

Antibodies to the immune inhibitory checkpoint axes PD-1/PD-L1 have proven particularly effective in cancer immunotherapy, inducing objective responses (Complete and Partial Responses) in ˜20% of patients—a significant improvement over standard of care (Carretero-Gonzalez, A. et al (2018) Oncotarget 9(9): 8706-8715). As evidenced by response rates, currently available anti-PD-1/PD-L1 antibodies are, however, active in only a minority of patients. Further, a fraction of initially responding patients will eventually develop resistance, and can no longer benefit from treatment. As such, mechanisms of unresponsiveness and resistance to PD-1/PD-L1 antibodies are a clinically important problem. Identifying and overcoming mechanisms of resistance to PD-1/PD-L1 antibodies is a major challenge, and opportunity, to improve cancer patient survival to this clinically important drug class.

Further, it is generally accepted that predictive biomarkers to identify patients most likely to respond to anti-PD-1/PD-L1 checkpoint blockade is an important strategy to identify patients likely to respond to therapy. It is equally important to, conversely, prevent unnecessary treatment of patients with these drugs that are frequently associated with significant, occasionally fatal, tolerability issues. Owing to their high cost, these therapies further present a significant burden to payers and health care systems. Examples of existing predictive biomarkers of clinical significance to anti-PD-1/PD-L1 antibody therapy are Micro Satellite Instability (MSI) (Le, D. T. et al (2015) N Engl J Med 372(26): 2509-2520; Le, D. T. et al (2017) Science 357(6349): 409-413), tumor mutational burden (Gubin, M. M. et al (2014) Nature 515(7528): 577-581; Snyder, A., et al (2014) N Engl J Med 371(23): 2189-2199; Tran, E. et al (2014) Science 344(6184): 641-645, Tran, E. et al (2015) Science 350(6266): 1387-1390), and tumor PD-L1 expression (Gibney, Weiner et al. 2016, Topalian, Taube et al. 2016).), tumor mutational burden, and tumor PD-L1 expression.

Fc gamma receptors (FcγRs) are membrane proteins which are found on the cell surface of immune effector cells including monocytes, macrophages, dendritic cells, neutrophils, mast cells, basophils, eosinophils and Natural Killer cells and B lymphocytes.

The name is derived from their binding specificity for the Fc region of antibodies. Fc receptors are found on the cell membrane—otherwise known as the plasma membrane or cytoplasmic membrane. FcγRs can be subdivided into activating FcγR and inhibitory FcγR, which are known to coordinately regulate cellular activation through binding of clustered immunoglobulin G Fc's, and transmission of activating or inhibitory signals into the cell through intracellular ITAM or ITIM motifs, respectively. FcγR binding of clustered immunoglobulin or immune complexes, can mediate antibody internalization into the cell, and can result in antibody-mediated phagocytosis, antibody-dependent cell-mediated cytotoxicity, or antigen presentation or cross-presentation. FcγRs are also known to mediate or enhance cross-linking of antibody-bound cell surface receptors. Such cross-linking is known to be required for some (Li, F. et al (2011) Science 333(6045): 1030-1034; White, A. L. et al (2011) J Immunol 187(4): 1754-1763) but not all (Richman, L. P. et al (2014) Oncoimmunology 3: e28610) antibodies ability to activate signaling in targeted cells, and may or may not be required to achieve therapeutic effects.

In humans, FcγRIIb (CD32b) is an inhibitory Fcγ receptor, while FcγRI (CD64), FcγRIIa (CD32a), FcγRIIc (CD32c) and FcγRIIIa (CD16a) are activating Fcγ receptors. FcγgRIIIb is a GPI-linked receptor expressed on neutrophils lacks an ITAM motif, and is thought to act as a decoy receptor that counterbalances activating FcγR signaling (Treffers, L. W. et al (2018) Front Immunol 9: 3124). In mice, the activating receptors are FcγRI, FcγRIII and FcγRIV.

It is well-known that antibodies can modulate immune cell activity through interaction with Fcγ receptors. Specifically, how antibody immune complexes modulate immune cell activation is determined by their relative engagement of activating and inhibitory Fcγ receptors. Different antibody isotypes bind with different affinity to activating and inhibitory Fcγ receptors, resulting in different A:I ratios (activation:inhibition ratios) (Nimmerjahn et al; Science. 2005 Dec. 2; 310(5753):1510-2).

By binding to an inhibitory Fcγ receptor through its Fc domain, an antibody can inhibit, block and/or down-modulate effector cell functions. By binding to an inhibitory FcγR through its Fc domain, antibodies can stimulate cell activation through aggregation of antibody-targeted signaling receptors on a target cell (Li, F. et al (2011) Science 333(6045): 1030-1034; White, A. L. et al (2011) J Immunol 187(4): 1754-1763; White, A. L. et al (2011) J Immunol 187(4): 1754-1763 White, A. L. et al (2014) J Immunol 193(4): 1828-1835).

By binding to an activating Fcγ receptor, an antibody can activate effector cell functions and thereby trigger mechanisms such as antibody-dependent cellular cytotoxicity (ADCC), antibody dependent cellular phagocytosis (ADCP), cytokine release, and/or antibody dependent endocytosis, as well as NETosis (i.e. activation and release of NETs, Neutrophil extracellular traps) in the case of neutrophils. Antibody binding to an activating Fcγ receptor can also lead to an increase in certain activation markers, such as CD40, MHCII, CD38, CD80 and/or CD86.

Consistent with concerted regulation of antibody-induced effector cell responses by activating and inhibitory Fcγ receptors, activating Fcγ receptors have been shown to promote tumor cell depletion and therapeutic activity of tumor-direct-targeting antibodies. Preclinical and clinical studies have demonstrated that the antitumor activity of tumor direct-targeting antibodies i.e. antibodies whose therapeutic activity involves direct binding and killing of tumor cells, e.g. anti-CD20, anti-Her2 and anti-EGFR antibodies, is enhanced in patients carrying higher affinity alleles for activating Fcγ receptors (Cartron, G. et al (2002) Blood 99(3): 754-758; Musolino, A., et al (2008) J Clin Oncol 26(11): 1789-1796; Zhang, W. et al (2007) J Clin Oncol 25(24): 3712-3718) and with antibody isotypes and formats that show stronger binding to activating compared with inhibitory Fcγ receptors (High A:I ratio) (Goede, V. et al (2014) N Engl J Med 370(12): 1101-1110). Conversely, therapeutic activity of tumor-direct targeting antibodies is enhanced in animals that lack the inhibitory FcγRIIB (Clynes, R. A. et al (2000) Nat Med 6(4): 443-446), or as more recently shown by some of the present inventors, when the inhibitory FcγRIIB is blocked by antagonistic anti-FcγRIIB antibodies (Roghanian, A. et al (2015) Cancer Cell 27(4): 473-488).

Emerging preclinical and clinical data demonstrate that the Fcγ receptors control also efficacy of immune modulatory antibodies, including immune checkpoint inhibitory antibodies targeting CTLA-4, PD-1/PD-L1. In humans and in mice, there is evidence that anti-CTLA-4 antibodies' therapeutic activity is promoted by engagement of activating Fcγ receptors; Melanoma patients carrying a high affinity allele of FcγRIIIa gene showed improved survival in response to ipilimumab compared with patients expressing lower affinity FcγRIIIa alleles. Moreover, in mice humanized for activating and inhibitory receptors, therapeutic efficacy was shown to be FcγR-dependent and was enhanced with antibody isotypes with high A:I ratio (Arce Vargas, F. et al (2018) Cancer Cell 33(4): 649-663 e644).

These findings prompted us to investigate the ability of FcγRIIB-blocking antibodies to enhance anti-CTLA-4 antibodies' activity. Two different types of FcγRIIB-blocking antibody have previously been generated and disclosed by some of the present inventors (Roghanian, A., et al (2015) Cancer Cell 27(4): 473-488); a human IgG1 that is proficient in binding both activating and inhibitory human FcγRs, and an Fc-engineered variant that shows severely impaired binding to FcγRs through its Fc domain. These two different types of anti-FcγRIIB antibody were shown to be equally antagonistic in blocking CD20 internalization and FcγRIIB signaling in B cells and both improved anti-CD20 mAb mediated B cell depletion in animals transgenic animals expressing human FcγRIIB and human CD20.

In International patent application No. PCT/EP2019/050566 we demonstrated that only anti-FcγRIIB antibodies that lacked Fc region, or whose Fc-region showed reduced or impaired binding to FcγRs, were able to enhance the therapeutic activity of anti-CTLA-4 antibodies' therapeutic activity. In different mouse experimental models of solid cancer, co-treatment with Fc:FcγR-binding-impaired anti-FcγRIIB antibody, but not Fc:FcγR-binding proficient anti-FcγRIIB, enhanced therapeutic activity of anti-CTLA-4, and in a humanized PBMC in vivo model demonstrated boosted Treg depletion of the clinically relevant anti-CTLA-4 antibody ipilimumab. Similar enhancing effects on depleting and/or therapeutic efficacy of antibodies specific for IL-2R (CD25) and PD-L1 were observed following combined treatment with the Fc:FcγR-binding impaired anti-FcγRIIB antibody, demonstrating that boosting effects were not restricted to a particular target or cell type.

A role for Fcγ receptors in controlling anti-PD-1/PD-L1 antibodies' therapeutic activity has been indicated by preclinical studies, but the individual role of activating and inhibitory Fcγ receptors has not been made clear. Dahan et al reported that mouse antibodies to PD-L1 benefit from FcγR-engagement for antitumor activity, but that conversely anti-PD-1 antibodies' activity is compromised by FcγR-engagement (Dahan, R. et al (2015) Cancer Cell 28(3): 285-295). Notably, anti-PD-1 antibody isotypes (mIgG2a) with a high A:I ratio i.e. delivering strong engagement of activating compared with inhibitory Fcγ receptors showed less therapeutic activity compared with the mIgG1 isotype with lower A:I ratio i.e. relatively stronger engagement of inhibitory Fcγ receptors and compared with anti-PD-1 variant antibodies defective for Fc:FcγR-binding.

Using clinically relevant human anti-PD-1 IgG4 antibody nivolumab, and a surrogate rat IgG2a antibody with claimed similar Fcγ receptor binding profile, Arlaukas and co-workers similarly found that Fc:FcγR-binding impaired (deglycosylated) anti-PD-1 variant antibodies had improved therapeutic activity compared with their Fc:FcγR-proficient wild-type human IgG4 and rat IgG2a anti-PD-1 counterparts (Arlauckas, S. P. et al (2017) Sci Transl Med 9(389)). Contrary to Dahan et al, however, using blocking antibodies to individual Fcγ receptors, the authors identified activating mouse Fcγ receptor III and inhibitory mouse Fcγ receptor II, as underlying the reduced anti-PD-1 antibody efficacy. Consequently, the relative importance of (individual) activating and inhibitory Fcγ receptors underlying the reduced efficacy of anti-PD-1 antibodies, how they limit efficacy of clinically relevant human anti-PD-1 antibodies, or which activating or inhibitory FcγRs should be blocked to enhance anti-PD-1 antibodies activity, is not clear from the prior art.

### SUMMARY OF THE INVENTION

Disclosed herein is a combination of:

a first antibody molecule that specifically binds FcγRIIb via its Fab region, and that binds an Fcγ receptor via its Fc region, and

a second antibody molecule that specifically binds to PD-1 and that binds to at least one Fcγ receptor via its Fc region;

for use in the treatment of cancer in a patient having tumor infiltrating T lymphocytes with a medium or high PD-1 expression.

Disclosed herein is also a pharmaceutical composition comprising:

(i) a first antibody molecule that specifically binds FcγRIIb via its Fab region, and that binds an Fcγ receptor via its Fc region, and

(ii) a second antibody molecule that specifically binds to PD-1 and that binds to at least one Fcγ receptor via its Fc region;

for use in the treatment of cancer in a patient having tumor infiltrating T lymphocytes with a medium or high PD-1 expression.

Disclosed herein is further a kit for use in the treatment of cancer in a patient having tumor infiltrating T lymphocytes with a medium or high PD-1 expression comprising:

(i) a first antibody molecule that specifically binds FcγRIIb via its Fab region, and that binds an Fcγ receptor via its Fc region, and

(ii) a second antibody molecule that specifically binds to PD-1 and that binds to at least one Fcγ receptor via its Fc region.

Further disclosed herein is the use of:

(i) a first antibody molecule that specifically binds FcγRIIb via its Fab region, and that binds an Fcγ receptor via its Fc region, and

(ii) a second antibody molecule that specifically binds to PD-1 and that binds to at least one Fcγ receptor via its Fc region;

in the manufacture of a medicament for use in the treatment of cancer in a patient having tumor infiltrating T lymphocytes with a medium or high PD-1 expression

Disclosed herein is also a method for treatment of cancer in a patient having tumor infiltrating T lymphocytes with a medium or high PD-1 expression, comprising administering:

(i) a first antibody molecule that specifically binds FcγRIIb via its Fab region, and that binds an Fcγ receptor via its Fc region, and

(ii) a second antibody molecule that specifically binds to PD-1 and that binds to at least one Fcγ receptor via its Fc region.

Disclosed herein is further a diagnostic test for determining if a patient will benefit from combined treatment with:

(i) a first antibody molecule that specifically binds FcγRIIb via its Fab region, and that binds an Fcγ receptor via its Fc region, and

(ii) a second antibody molecule that specifically binds to PD-1 and that binds to at least one Fcγ receptor via its Fc region, which test comprises determining the PD-1 expression on the patient's tumor infiltrating T lymphocytes, wherein medium or high PD-1 expression indicates that the patient will benefit from combined treatment. Correspondingly, lack of medium or high PD-1 expression on T lymphocytes while low PD-1 expression indicates that the patient will not benefit from combined treatment.

### DETAILED DESCRIPTION OF THE INVENTION

Here we demonstrate that ONLY an Fc:FcγR-binding proficient, and not Fc:FcγR-binding impaired, anti-FcγRIIB antibody enhances therapeutic efficacy of anti-PD-1 antibodies in vivo, and prevents phagocytosis induced by clinically relevant human anti-PD-1 antibodies of PD-1 high-expressing T cells in vitro. This finding is novel, and unexpected, since above referenced studies on the role of FcγRs in anti-PD-1 therapy indicated either a broad role for activating compared with inhibitory FcγRs (Dahan, R. et al (2015) Cancer Cell 28(3): 285-295), or individual activating (FcγRIII) and the inhibitory FcγRIIB (Arlauckas, S. P. et al (2017) Sci Transl Med 9(389)), as underlying impaired anti-PD-1 antibody activity.

The present invention is further surprising in view of some of the present inventors' earlier findings relating to antibodies to other immune checkpoints, notably including anti-CTLA-4, where only Fc:FcγR-binding impaired, and not Fc:FcγR-binding proficient, anti-FcγRIIB antibody enhances therapeutic activity.

Thus, the present invention concerns the combined use of:

(i) an antibody molecule that specifically binds FcγRIIb via its Fab region, and that binds an Fcγ receptor via its Fc region (herein denoted the first antibody molecule), and

(ii) an antibody molecule that specifically binds to PD-1 and that binds to at least one Fcγ receptor via its Fc region (herein denoted the second antibody molecule)

in the treatment of cancer in a patient having tumor infiltrating T lymphocytes with a medium or high PD-1 expression

This combination is intended to be used in the treatment of a cancer, such as a solid cancer, in a patient, with the aim to improve therapeutic efficacy of the antibody molecule that binds specifically to PD-1, i.e. the anti-PD-1 antibody, through diminished binding to FcγRs, including FcγRIIB.

The antibody molecule according to the invention that specifically binds FcγRIIb, i.e. the first antibody, binds to or interacts with this Fcγ receptor via the Fab region of the antibody, i.e. via the antigen-binding region on an antibody that binds to antigens which is composed of one constant and one variable domain of each of the heavy and the light chain. In particular, it binds to FcγRIIb present on an immune effector cell e.g. a macrophage, and in particular to FcγRIIb present on the surface of an immune effector cell.

In addition to the above, the antibody molecule according to the invention that specifically binds FcγRIIb, i.e. the first antibody molecule, also binds to activating Fcγ receptor through interaction between the Fc region and Fc receptor, as is known and has been extensively characterized for antibodies of human IgG1 isotype (Bruhns, P. et al (2009) Blood 113(16): 3716-3725).

This has at least the following therapeutically important consequence: both activating and inhibitory Fcγ receptors are blocked in an anti-FcγRIIB antibody-dependent (Fab- and Fc-) manner, preventing macrophage phagocytosis or other Fcγ receptor expressing immune effector cells' anti-PD-1-antibody-mediated elimination of anti-PD-1 antibody coated anti-tumor T cells (coated in this context means that the anti-PD-1 antibody has bound to the cells). The mechanism may additionally involve inhibition of macrophage FcγR-dependent transfer of PD-1 antibodies from T cells to FcγR-expressing effector cells e.g. macrophages, as has been previously described (Arlauckas, S. P. et al (2017) Sci Transl Med 9(389)).

Fc gamma receptor expressing immune effector cell refers herein to principally innate effector cells, and includes specifically macrophages, neutrophils, monocytes, natural killer (NK) cells, basophils, eosinophils, mast cells, and platelets. Cytotoxic T cells and memory T cells do not typically express FcγRs, but may do so under specific circumstances. In some embodiments the immune effector cell is an innate immune effector cell. In some embodiments, the immune effector cell is a macrophage.

The antibody molecule that specifically binds to or interacts with PD-1, i.e. the second antibody molecule, has an Fc region that binds to or interacts with an activating Fcγ receptor that permits antibody-PD-1 antibody dependent FcγR effector cell dependent elimination of anti-PD-1 antibody coated antitumor T cells. The immune cell to which the anti-PD-1 antibody molecule binds is an immune cell that confers critical antitumor activity, such as a CD8+ or CD4+ T cell.

Consequently, any anti-PD-1 variant antibody, including those of human IgG4, IgG1, IgG2 and IgG3 isotypes, whose Fc region binds to or interacts with an activating Fcγ receptor to an extent that results in FcγR expressing effector cell elimination of the PD-1 expressing antitumor T cell, can be combined with an Fc:FcγR-binding proficient anti-FcγRIIB antibody, i.e. with an antibody molecule that specifically binds FcγRIIb via its Fab region, and that binds an Fcγ receptor via its Fc region.

The second antibody is an anti-PD-1 antibody. PD-1 (programmed cell death protein 1) also known as CD279 is an immune checkpoint, i.e. a checkpoint protein on immune cells. It promotes apoptosis of antigen-specific T-cells in lymph nodes and reduces apoptosis in regulatory T cells. PD-1 inhibitors, such as nivolumab (OPDIVO®), pembrolizumab (KEYTRUDA®) and cemiplimab (LIBTAYO®), are used in cancer treatment to activate the immune system to attack tumors. Monoclonal antibodies that target either PD-1 or PD-L1 can block the binding of PD-1 to PD-L1, which may boost the immune response against cancer cells.

The anti-PD-1 antibody binds to PD-1 expressed on intratumoral T cells.

Patients that benefit from treatment in accordance with the present invention are patients who, per standard criteria for approved anti-PD-1 antibody containing regimen, are eligible for anti-PD-1 therapy and in addition who have tumor infiltrating T lymphocytes (i.e. tumor infiltrating CD3+ lymphocytes) with a medium or high PD-1 expression. Patients who are eligible for anti-PD-1 therapy include patients suffering from melanoma; lung cancer, including small cell lung cancer (SCLC) and non-small cell lung carcinoma (NSCLC) (including non-squamous NSCLC and squamous NSCLC, and including metastatic NSCLC); head and neck cancer, including head and neck squamous cell carcinoma (HNSCC); Hodgkin lymphoma; primary mediastinal B-cell lymphoma (PMBCL); bladder cancer, including advanced urothelial carcinoma; colorectal cancer, including cancer that is instability-high (MSI-H) and/or mismatch repair deficient (dMMR); gastric cancer, including advanced gastric cancer and gastric or gastroesophageal junction (GEJ) adenocarcinoma; cervical cancer; liver cancer, including hepatocellular carcinoma; Merkel cell carcinoma (MCC); kidney cancer, including renal cell carcinoma (RCC) and cutaneous squamous cell carcinoma (CSCC), including locally advanced CSCC in patients who are not candidates for curative surgery or curative radiation. The number of indications that can be treated is rapidly expanding with new trials, new anti-PD-1 antibodies, and new combinations, as will be known to a person trained in the art.

In the work leading to the present invention it was observed that when an anti-PD-1 antibody is administered to T cells that have a medium or high expression of PD-1 this may lead to phagocytosis of the anti-PD-1 antibody coated T-cells, and in particular of CD8 positive T-cells. In the work leading to the present invention, it was further found that by administering an antibody molecule that specifically binds FcγRIIb via its Fab region, and that binds an Fcγ receptor via its Fc region, together with the anti-PD-1 antibody, this phagocytosis could be blocked for T cells that have a medium or high expression of PD-1. The in vivo relevance of the in vitro assay used for the above findings, which is further explained in the examples below, was confirmed in two different solid cancer experimental models comprising immune competent mice, where significantly enhanced survival of combined treatment with Fc:FcγR-binding proficient anti-FcγRIIB and anti-PD-1 antibodies compared to single agent treatment with anti-PD-1, was observed. This is also shown in more detail in the examples below. Further supporting the in vivo relevance of the in vitro assay, intratumoral in vivo T cell PD-1 expression levels were similar in the two settings; in vivo intratumoral T cell PD-1 expression ranged from ˜20,000 to 80,000 PD-1 molecules per cell, spanning in vitro expression levels of PD-1 medium (15,500 to 78,000 PD-1 molecules) and high expressing (65,000 to 391,000 PD1 molecules per cell) human T cells, both of which were sensitive to Fc:FcγR-binding proficient anti-FcγRIIB block of human anti-PD-1-mediated phagocytosis.

Thus, in the context of the present invention, patients that may benefit from the treatment described herein are patients that have at least 10% tumor infiltrating T lymphocytes having a medium or high expression of PD-1.

Herein, medium or high expression refers to an expression of >15,500 PD-1 molecules per cell for at least 10% of the tumor infiltrating T lymphocytes. As explained further below, the absolute numbers of PD-1 expression may vary depending on what anti-PD-1 antibody and/or what method is used for measuring the PD-1 expression. Thus, a medium or high expression of equal to or above 15,500 PD-1 molecules per cell as used herein is measured with the method described herein, and/or using the anti-human PD-1 antibody EH12.2H7 (obtainable from BioLegend).

Similar to the in vivo setting, an individual patient's intratumoral T cells will show heterogenous PD-1 expression. An individual patient may have different cell populations having different expression of PD-1, such as one population having low expression and one population having medium or high expression. In some embodiments, at least 15% of the patient's tumor infiltrating CD3+T lymphocytes have a medium or high PD-1 expression. In some embodiments, at least 20% of the patient's tumor infiltrating CD3+T lymphocytes have a medium or high PD-1 expression. In some embodiments, at least 25% of the patient's tumor infiltrating CD3+T lymphocytes have a medium or high PD-1 expression. In some embodiments, at least 30% of the patient's tumor infiltrating CD3+T lymphocytes have a medium or high PD-1 expression. In some embodiments, at least 35% of the patient's tumor infiltrating CD3+T lymphocytes have a medium or high PD-1 expression. In some embodiments, at least 40% of the patient's tumor infiltrating CD3+T lymphocytes have a medium or high PD-1 expression. In some embodiments, at least 45% of the patient's tumor infiltrating CD3+T lymphocytes have a medium or high PD-1 expression. In some embodiments, at least 50% of the patient's tumor infiltrating CD3+T lymphocytes have a medium or high PD-1 expression. In some embodiments, at least 55% of the patient's tumor infiltrating CD3+T lymphocytes have a medium or high PD-1 expression. In some embodiments, at least 60% of the patient's tumor infiltrating CD3+T lymphocytes have a medium or high PD-1 expression. In some embodiments, at least 65% of the patient's tumor infiltrating CD3+T lymphocytes have a medium or high PD-1 expression. In some embodiments, at least 70% of the patient's tumor infiltrating CD3+T lymphocytes have a medium or high PD-1 expression. In some embodiments, at least 75% of the patient's tumor infiltrating CD3+T lymphocytes have a medium or high PD-1 expression. In some embodiments, at least 80% of the patient's tumor infiltrating CD3+T lymphocytes have a medium or high PD-1 expression. In some embodiments, at least 85% of the patient's tumor infiltrating CD3+T lymphocytes have a medium or high PD-1 expression. In some embodiments, at least 90% of the patient's tumor infiltrating CD3+T lymphocytes have a medium or high PD-1 expression.

In some embodiments, it is the patient's tumor infiltrating CD3 positive and CD8 positive (CD3+CD8+) T lymphocytes that have a medium or high PD-1 expression.

In some embodiments, at least 10% of the patient's tumor infiltrating CD3+CD8+T lymphocytes have a medium or high PD-1 expression. In some embodiments, at least 15% of the patient's tumor infiltrating CD3+CD8+T lymphocytes have a medium or high PD-1 expression. In some embodiments, at least 20% of the patient's tumor infiltrating CD3+CD8+T lymphocytes have a medium or high PD-1 expression. In some embodiments, at least 25% of the patient's tumor infiltrating CD3+CD8+T lymphocytes have a medium or high PD-1 expression. In some embodiments, at least 30% of the patient's tumor infiltrating CD3+CD8+T lymphocytes have a medium or high PD-1 expression. In some embodiments, at least 35% of the patient's tumor infiltrating CD3+CD8+T lymphocytes have a medium or high PD-1 expression. In some embodiments, at least 40% of the patient's tumor infiltrating CD3+CD8+T lymphocytes have a medium or high PD-1 expression. In some embodiments, at least 45% of the patient's tumor infiltrating CD3+CD8+T lymphocytes have a medium or high PD-1 expression. In some embodiments, at least 50% of the patient's tumor infiltrating CD3+CD8+T lymphocytes have a medium or high PD-1 expression. In some embodiments, at least 55% of the patient's tumor infiltrating CD3+CD8+T lymphocytes have a medium or high PD-1 expression. In some embodiments, at least 60% of the patient's tumor infiltrating CD3+CD8+T lymphocytes have a medium or high PD-1 expression. In some embodiments, at least 65% of the patient's tumor infiltrating CD3+CD8+T lymphocytes have a medium or high PD-1 expression. In some embodiments, at least 70% of the patient's tumor infiltrating CD3+CD8+T lymphocytes have a medium or high PD-1 expression. In some embodiments, at least 75% of the patient's tumor infiltrating CD3+CD8+T lymphocytes have a medium or high PD-1 expression. In some embodiments, at least 80% of the patient's tumor infiltrating CD3+CD8+T lymphocytes have a medium or high PD-1 expression. In some embodiments, at least 85% of the patient's tumor infiltrating CD3+CD8+T lymphocytes have a medium or high PD-1 expression. In some embodiments, at least 90% of the patient's tumor infiltrating CD3+CD8+T lymphocytes have a medium or high PD-1 expression.

The expression of PD-1 on the tumor cells of an individual patient can be measured using tumor biopsy-derived cells or tissue. More specifically, absolute T cell expression levels can be quantified using herein described, or equivalent, flow-cytometry and bead-based antibody and cell epitope quantitation kit(s). Alternatively, semiquantitative analyses can be performed by immunohistochemistry using tumor tissue biopsies, comparing anti-PD-1 staining of patient biopsies to that of tissue or cytospun cells, expressing defined and determined PD-1 levels associated with sensitivity (>15,500 PD-1 molecules per cell), or no sensitivity (<15,500 PD-1 molecules per cell) to anti-FcγRIIB-mediated boosting of anti-PD-1 antibody activity. Importantly, if the method to determine human T cell PD-1 expression is different to herein described quantification method, or uses a different anti-PD-1 antibody clone or fluorescence labeling, the assay needs to be compared and validated, e.g. to the method described in detail in the examples below, in particular in Example 1 with reference to FIG. 1. In general terms, one way of quantifying PD-1 expression, as exemplified in Example 1 with reference to FIG. 1, is to use antibody labeled with fluorochrome at a defined ratio; for example—and as preferably in some embodiments—using antibody labeled with phycoerythrin (PE) at a 1:1 ratio. Thereby, using beads with defined numbers of fluorochrome (e.g. PE) molecules to generate a standard curve, the number of molecules of antibody bound to a cell can be determined. The cells to be tested are incubated with labeled anti-PD1 antibody (such as the anti-human PD-1 antibody EH12.2H7 from BioLegend) and analyzed using a FACs machine set up in such a way that beads and cells can be run on the same settings. A standard curve is generated, for example by plotting Log molecules per bead versus Log fluorescence, and then the fluorochrome labeled anti-PD1 antibody stained cells are run, and the Log mean fluorescence intensity (MFI) is used to calculate the number of antibodies bound.

As mentioned above, the absolute numbers may vary depending on what anti-PD1 antibody is used for the measurement.

In addition to binding specifically to PD-1 on the immune cell, the second antibody molecule binds to at least one Fcγ receptor via its Fc region. In some embodiments, the second antibody molecule binds to at least one activating Fcγ receptor via its Fc region. The second antibody may be capable of binding, via its Fc region, to an activating Fcγ receptor, such as an activating Fcγ receptor, present on an immune effector cell. In order to be able to bind to an activating Fcγ receptor, the Fc region of the second antibody may, at least in some embodiments, be glycosylated at position 297. The carbohydrate residue in this position helps binding to Fcγ receptors. In some embodiments it is preferred that these residues are biantennary carbohydrates which contain GlnNAc, mannose, with terminal galactose residues and sialic acid. It should contain the CH₂ part of the Fc molecule.

The present invention further relates to a diagnostic test that can be used to identify patients that benefit from the treatment described herein, i.e. combined treatment with (i) a first antibody molecule that specifically binds FcγRIIb via its Fab region, and that binds an Fcγ receptor via its Fc region, and (ii) a second antibody molecule that specifically binds to PD-1 and that binds to at least one Fcγ receptor via its Fc region. Based on in vivo PD1 expression levels associated with anti-FcγRIIB-mediated enhanced anti-PD-1 antibody efficacy, and an in vitro phagocytosis assay incorporating therapeutically relevant human anti-PD-1 antibody, and human T cells and macrophages, the inventors have determined that certain T cell PD-1 receptor expression levels are associated with, and needed for, anti-FcγRIIB-mediated boosting of anti-PD-1 therapeutic efficacy. The diagnostic test according to the invention is based on this finding, and accordingly comprises measurement of the expression of PD-1 on the tumor cells in a sample, such as tumor biopsy-derived cells or tissue, obtained from a patient. An absolute or a semi-quantitative analysis of PD1 expression levels on T cells can be used, as described above. In some embodiments, the diagnostic test is based on the use of the anti-PD1 antibody EH12.2H7 for measurement of the PD-1 expression; expression of at least 15,500 PD-1 molecules per T lymphocyte predicts that the patient may benefit from combined treatment according to the invention, as further described above and in Example 1.

Antibodies are well known to those skilled in the art of immunology and molecular biology. Typically, an antibody comprises two heavy (H) chains and two light (L) chains. Herein, we sometimes refer to this complete antibody molecule as a full-size or full-length antibody. The antibody's heavy chain comprises one variable domain (VH) and three constant domains (CH1, CH2 and CH3), and the antibody's molecule light chain comprises one variable domain (VL) and one constant domain (CL). The variable domains (sometimes collectively referred to as the Fᵥ region) bind to the antibody's target, or antigen. Each variable domain comprises three loops, referred to as complementary determining regions (CDRs), which are responsible for target binding. The constant domains are not involved directly in binding an antibody to an antigen, but exhibit various effector functions. Depending on the amino acid sequence of the constant region of their heavy chains, antibodies or immunoglobulins can be assigned to different classes. There are five major classes of immunoglobulins: IgA, IgD, IgE, IgG and IgM, and in humans several of these are further divided into subclasses (isotypes), e.g., IgG1, IgG2, IgG3, and IgG4; IgA1 and IgA2.

Another part of an antibody is the Fc region (otherwise known as the fragment crystallizable domain), which comprises two of the constant domains of each of the antibody's heavy chains. As mentioned above, the Fc region is responsible for interactions between the antibody and Fc receptor.

The term antibody molecule, as used herein, encompasses full-length or full-size antibodies as well as functional fragments of full length antibodies and derivatives of such antibody molecules.

Functional fragments of a full-size antibody have the same antigen binding characteristics as the corresponding full-size antibody and include either the same variable domains (i.e. the VH and VL sequences) and/or the same CDR sequences as the corresponding full-size antibody. That the functional fragment has the same antigen binding characteristics as the corresponding full-size antibody means that it binds to the same epitope on the target as the full-size antibody. Such a functional fragment may correspond to the Fv part of a full-size antibody. Alternatively, such a fragment may be a Fab, also denoted F(ab), which is a monovalent antigen-binding fragment that does not contain a Fc part, or a F(ab′)₂, which is an divalent antigen-binding fragment that contains two antigen-binding Fab parts linked together by disulfide bonds, or a F(ab′), i.e. a monovalent-variant of a F(ab′)₂. Such a fragment may also be single chain variable fragment (scFv).

A functional fragment does not always contain all six CDRs of a corresponding full-size antibody. It is appreciated that molecules containing three or fewer CDR regions (in some cases, even just a single CDR or a part thereof) are capable of retaining the antigen-binding activity of the antibody from which the CDR(s) are derived. For example, in Gao et al., 1994, J. Biol. Chem., 269: 32389-93 it is described that a whole VL chain (including all three CDRs) has a high affinity for its substrate.

Molecules containing two CDR regions are described, for example, by Vaughan & Sollazzo 2001, Combinatorial Chemistry & High Throughput Screening, 4: 417-430. On page 418 (right column—3 Our Strategy for Design) a minibody including only the H1 and H2 CDR hypervariable regions interspersed within framework regions is described. The minibody is described as being capable of binding to a target. Pessi et al., 1993, Nature, 362: 367-9 and Bianchi et al., 1994, J. Mol. Biol., 236: 649-59 are referenced by Vaughan & Sollazzo and describe the H1 and H2 minibody and its properties in more detail. In Qiu et al., 2007, Nature Biotechnology, 25:921-9 it is demonstrated that a molecule consisting of two linked CDRs are capable of binding antigen. Quiocho 1993, Nature, 362: 293-4 provides a summary of “minibody” technology. Ladner 2007, Nature Biotechnology, 25:875-7 comments that molecules containing two CDRs are capable of retaining antigen-binding activity.

Antibody molecules containing a single CDR region are described, for example, in Laune et al., 1997, JBC, 272: 30937-44, in which it is demonstrated that a range of hexapeptides derived from a CDR display antigen-binding activity and it is noted that synthetic peptides of a complete, single, CDR display strong binding activity. In Monnet et al., 1999, JBC, 274: 3789-96 it is shown that a range of 12-mer peptides and associated framework regions have antigen-binding activity and it is commented on that a CDR3-like peptide alone is capable of binding antigen. In Heap et al., 2005, J. Gen. Virol., 86: 1791-1800 it is reported that a “micro-antibody” (a molecule containing a single CDR) is capable of binding antigen and it is shown that a cyclic peptide from an anti-HIV antibody has antigen-binding activity and function. In Nicaise et al., 2004, Protein Science, 13:1882-91 it is shown that a single CDR can confer antigen-binding activity and affinity for its lysozyme antigen.

Thus, antibody molecules having five, four, three or fewer CDRs are capable of retaining the antigen binding properties of the full-length antibodies from which they are derived.

The antibody molecule may also be a derivative of a full-length antibody or a fragment of such an antibody. When a derivative is used it should have the same antigen binding characteristics as the corresponding full-length antibody in the sense that it binds to the same epitope on the target as the full-length antibody.

Thus, by the term “antibody molecule”, as used herein, we include all types of antibody molecules and functional fragments thereof and derivatives thereof, including: monoclonal antibodies, polyclonal antibodies, synthetic antibodies, recombinantly produced antibodies, multi-specific antibodies, bi-specific antibodies, human antibodies, antibodies of human origin, humanized antibodies, chimeric antibodies, single chain antibodies, single-chain Fvs (scFv), Fab fragments, F(ab′)₂ fragments, F(ab′) fragments, disulfide-linked Fvs (sdFv), antibody heavy chains, antibody light chains, homo-dimers of antibody heavy chains, homo-dimers of antibody light chains, heterodimers of antibody heavy chains, heterodimers of antibody light chains, antigen binding functional fragments of such homo- and heterodimers.

Further, the term “antibody molecule”, as used herein, includes all classes of antibody molecules and functional fragments, including: IgG, IgG1, IgG2, IgG3, IgG4, IgA, IgM, IgD, and IgE, unless otherwise specified.

In some embodiments, the first antibody is a human IgG1. The skilled person will appreciate that the mouse IgG2a and human IgG1 engage and are capable of blocking activatory Fc gamma receptors, thereby preventing anti-PD-1 antibody mediated engagement of activating Fc gamma receptors on immune effector cells and their subsequent elimination of anti-PD-1 antibody coated effector T cell by e.g. ADCP or ADCC. As such, in embodiments where the mouse IgG2a is the preferred isotype for deletion in the mouse, human IgG1 is a preferred isotype for deletion in human in such embodiments.

In some embodiments, the first antibody is a human IgG1. In other embodiments, the first antibody is a human IgG4, IgG3 or IgG2. In other embodiments, the first antibody is a human IgG antibody Fc-engineered for enhanced binding to Fc gamma receptors. In some embodiments the human IgG antibody is Fc-engineered for improved binding to one or several activating Fcγ receptors and/or engineered for improved relative binding to activating over inhibitory Fcγ receptors. In some embodiments, the anti-FcγRIIB antibody is an Fc-engineered human IgG antibody. Examples of such engineered antibody variants include afucosylated antibodies with selective improved antibody binding to FcγRIIIA, and antibodies engineered by directed, mutational, or by other means, amino acid substitution resulting in improved binding to one or several activating Fcγ receptors compared to inhibitory FcγRIIB (Richards et al. 2008. ‘Optimization of antibody binding to FcgammaRIIa enhances macrophage phagocytosis of tumor cells’, Mol Cancer Ther, 7: 2517-27; Lazar et al. 2006. ‘Engineered antibody Fc variants with enhanced effector function’, Proc Natl Acad Sci USA, 103: 4005-10) In some embodiments, the human IgG antibody that is engineered for improved binding to activating Fc gamma receptors may be a human IgG antibody carrying the two mutations S239D and 1332E, or the three mutations S239D, 1332E and A330L, and/or G236A mutations in its Fc portion. In some embodiments, the human IgG antibody that is engineered for improved binding to activating Fc gamma receptors may be an afucosylated human IgG antibody.

In some embodiments, the second antibody is a human IgG4, the isotype of currently approved anti-PD-1 antibodies nivolumab, pembrolizumab and ceplizumab by the FDA. The skilled person will appreciate that the several murine antibody isotypes are capable of binding both activating and inhibitory Fc gamma receptors. Importantly, the skilled person will know that the rat IgG2a isotype binding to mouse activating and inhibitory Fc gamma receptors is known to closely mimick human IgG4 isotype binding to human activating and inhibitory Fc gamma receptors (Arlauckas, S. P. et al (2017) Sci Transl Med 9(389)). The skilled person will further know that besides human isotypes IgG1 and IgG4, human IgG3 and IgG2 antibodies may productively engage with human FcγRs (Sanders, L. A. et al (1995) Infect Immun 63(1): 73-81), and mediate antibody-dependent T cell depletion through e.g. ADCP and ADCC following activation of activating Fc gamma receptor bearing immune cells (Arce Vargas, F. et al (2018) Cancer Cell 33(4): 649-663 e644). Consequently, in some embodiments the second antibody may be a human IgG1 or IgG2 or IgG3 antibody.

As outlined above, different types and forms of antibody molecules are encompassed by the invention, and would be known to the person skilled in immunology. It is well known that antibodies used for therapeutic purposes are often modified with additional components which modify the properties of the antibody molecule.

Accordingly, we include that an antibody molecule of the invention or an antibody molecule used in accordance with the invention (for example, a monoclonal antibody molecule, and/or polyclonal antibody molecule, and/or bi-specific antibody molecule) comprises a detectable moiety and/or a cytotoxic moiety.

By “detectable moiety”, we include one or more from the group comprising of: an enzyme; a radioactive atom; a fluorescent moiety; a chemiluminescent moiety; a bioluminescent moiety. The detectable moiety allows the antibody molecule to be visualized in vitro, and/or in vivo, and/or ex vivo.

By “cytotoxic moiety”, we include a radioactive moiety, and/or enzyme, wherein the enzyme is a caspase, and/or toxin, wherein the toxin is a bacterial toxin or a venom; wherein the cytotoxic moiety is capable of inducing cell lysis.

We further include that the antibody molecule may be in an isolated form and/or purified form, and/or may be PEGylated. PEGylation is a method by which polyethylene glycol polymers are added to a molecule such as an antibody molecule or derivative to modify its behavior, for example to extend its half-life by increasing its hydrodynamic size, preventing renal clearance.

As discussed above, the CDRs of an antibody bind to the antibody target. The assignment of amino acids to each CDR described herein is in accordance with the definitions according to Kabat E A et al. 1991, In “Sequences of Proteins of Immunological Interest” Fifth Edition, NIH Publication No. 91-3242, pp xv-xvii.

As the skilled person would be aware, other methods also exist for assigning amino acids to each CDR. For example, the International ImMunoGeneTics information system (IMGT®) (http://www.imgt.org/ and Lefranc and Lefranc “The Immunoglobulin FactsBook” published by Academic Press, 2001).

In a further embodiment, the antibody molecule of the present invention or used according to the invention is an antibody molecule that is capable of competing with the specific antibodies provided herein, for example antibody molecules comprising any of the amino acid sequences set out in for example SEQ ID NOs: 1-194 for binding to the specific target.

By “capable of competing for” we mean that the competing antibody is capable of inhibiting or otherwise interfering, at least in part, with the binding of an antibody molecule as defined herein to the specific target.

For example, such a competing antibody molecule may be capable of inhibiting the binding of an antibody molecule described herein by at least about 10%; for example at least about 20%, or at least about 30%, at least about 40%, at least about 50%, at least about 60%, at least about 70%, at least about 80%, at least about 90%, at least about 95%, about 100% and/or inhibiting the ability of the antibody described herein to prevent or reduce binding to the specific target by at least about 10%; for example at least about 20%, at least about 30%, at least about 40%, at least about 50%, at least about 60%, at least about 70%, at least about 80%, at least about 90%, at least about 95%, or about 100%.

Competitive binding may be determined by methods well known to those skilled in the art, such as Enzyme-linked immunosorbent assay (ELISA).

ELISA assays can be used to evaluate epitope-modifying or blocking antibodies. Additional methods suitable for identifying competing antibodies are disclosed in Antibodies: A Laboratory Manual, Harlow & Lane, which is incorporated herein by reference (for example, see pages 567 to 569, 574 to 576, 583 and 590 to 612, 1988, CSHL, NY, ISBN 0-87969-314-2).

It is well known that an antibody specifically binds to or interacts with a defined target molecule or antigen, and that this means that the antibody preferentially and selectively binds its target and not a molecule which is not a target.

The targets of the antibodies according to the present invention, or of the antibodies used in accordance with the invention, are expressed on the surface of cells, i.e. they are cell surface antigen, which would include an epitope (otherwise known in this context as a cell surface epitope) for the antibody. Cell surface antigen and epitope are terms that would be readily understood by one skilled in immunology or cell biology.

By “cell surface antigen”, we include that the cell surface antigen is exposed on the extracellular side of the cell membrane, but may only be transiently exposed on the extracellular side of the cell membrane. By “transiently exposed”, we include that the cell surface antigen may be internalized into the cell, or released from the extracellular side of the cell membrane into the extracellular space. The cell surface antigen may be released from the extracellular side of the cell membrane by cleavage, which may be mediated by a protease.

We also include that the cell surface antigen may be connected to the cell membrane, but may only be transiently associated with the cell membrane. By “transiently associated”, we include that the cell surface antigen may be released from the extracellular side of the cell membrane into the extracellular space. The cell surface antigen may be released from the extracellular side of the cell membrane by cleavage, which may be mediated by a protease.

We further include that the cell surface antigen may be a peptide, or a polypeptide, or a carbohydrate, or an oligosaccharide chain, or a lipid; and/or an epitope that is present on a protein, or a glycoprotein, or a lipoprotein.

Methods of assessing protein binding are known to the person skilled in biochemistry and immunology. It would be appreciated by the skilled person that those methods could be used to assess binding of an antibody to a target and/or binding of the Fc region of an antibody to an Fc receptor; as well as the relative strength, or the specificity, or the inhibition, or prevention, or reduction in those interactions. Examples of methods that may be used to assess protein binding are, for example, immunoassays, BIAcore, western blots, radioimmunoassay (RIA) and enzyme-linked immunosorbent assays (ELISAs) (See Fundamental Immunology Second Edition, Raven Press, New York at pages 332-336 (1989) for a discussion regarding antibody specificity).

Accordingly, by “antibody molecule the specifically binds” or “target specific antibody molecule” we include that the antibody molecule specifically binds a target but does not bind to non-target, or binds to a non-target more weakly (such as with a lower affinity) than the target.

We also include the meaning that the antibody specifically binds to the target at least two-fold more strongly, or at least five-fold more strongly, or at least 10-fold more strongly, or at least 20-fold more strongly, or at least 50-fold more strongly, or at least 100-fold more strongly, or at least 200-fold more strongly, or at least 500-fold more strongly, or at least than about 1000-fold more strongly than to a non-target.

Additionally, we include the meaning that the antibody specifically binds to the target if it binds to the target with a K_{d} of at least about 10⁻¹ K_{d}, or at least about 10⁻² K_{d}, or at least about 10⁻³ K_{d}, or at least about 10⁻⁴ K_{d}, or at least about 10⁻⁵ K_{d}, or at least about 10⁻⁶ K_{d}, or at least about 10⁻⁷ K_{d}, or at least about 10⁻⁸ K_{d}, or at least about 10⁻⁹ K_{d}, or at least about 10⁻¹⁰ K_{d}, or at least about 10⁻¹¹ K_{d}, or at least about 10⁻¹² K_{d}, or at least about 10⁻¹³ K_{d}, or at least about 10⁻¹⁴ K_{d}, or at least about 10⁻¹⁵ K_{d}.

In some embodiments the antibody molecule that specifically binds FcγRIIb is a human antibody.

In some embodiments, the antibody molecule that specifically binds FcγRIIb is an antibody of human origin, i.e. an originally human antibody that has been modified as described herein.

In some embodiments, the antibody molecule that specifically binds FcγRIIb is a humanized antibody, i.e. an originally non-human antibody that has been modified to increase its similarity to a human antibody. The humanized antibodies may, for example, be of murine antibodies or lama antibodies.

In some embodiments, the antibody molecule that specifically binds FcγRIIb comprises the following constant regions (CH and CL):

IgG1-CH
[SEQ ID NO: 1]
ASTKGPSVFPLAPSSKSTSGGTAALGCLVKDYFPEPVTVSWNSGALTSGV
HTFPAVL-
QSSGLYSLSSVVTVPSSSLGTQTYICNVNHKPSNTKVDKKVEPKSCDKTH
TCPPC-
PAPELLGGPSVFLFPPKPKDTLMISRTPEVTCVVVDVSHEDPEVKFNWYV
DGV-
EVHNAKTKPREEQYNSTYRVVSVLTVLHQDWLNGKEYKCKVSNKALPAPI
EKTISK-
AKGQPREPQVYTLPPSRDELTKNQVSLTCLVKGFYPSDIAVEWESNGQPE
NNYK-
TTPPVLDSDGSFFLYSKLTVDKSRWQQGNVFSCSVMHEALHNHYTQKSLS
LSPGK
IgG1-CL
[SEQ ID NO: 2]
QPKAAPSVTLFPPSSEELQANKATLVCLISDFYPGAVTVAWKADSSPVKA
GVETTTP-
SKQSNNKYAASSYLSLTPEQWKSHRSYSCQVTHEGSTVEKTVAPTECS

In some embodiments, the antibody molecule that specifically binds FcγRIIb comprises one or more sequences of the following clones:

Antibody clone: 1A01
1A01-VH
[SEQ ID NO: 3]
EVQLLESGGGLVQPGGSLRLSCAASGFTFSDYYMNWIRQTPGKGLEWVSL
IGWDG-
GSTYYADSVKGRFTISRDNSENTLYLQMNSLRAEDTAVYYCARAYSGYEL
DYWGQ-
GTLVTVSS
1A01-VL
[SEQ ID NO: 27]
QSVLTQPPSASGTPGQRVTISCSGSSSNIGNNAVNWYQQLPGTAPKLLIY
DNNN-
RPSGVPDRFSGSKSGTSASLAISGLRSEDEADYYCAAWDDSLNASIFGGG
TKLTVLG
CDR regions
CDRH1:
[SEQ ID NO: 51]
DYYMN
CDRH2:
[SEQ ID NO: 52]
LIGWDGGSTYYADSVKG
CDRH3:
[SEQ ID NO: 53]
AYSGYELDY
CDRL1:
[SEQ ID NO: 54]
SGSSSNIGNNAVN
CDRL2:
[SEQ ID NO: 55]
DNNNRPS
CDRL3:
[SEQ ID NO: 56]
AAWDDSLNASI
Antibody clone: 1B07
1B07-VH
[SEQ ID NO: 4]
EVQLLESGGGLVQPGGSLRLSCAASGFTFSSYGMHWVRQAPGKGLEWVAF
TRYD-
GSNKYYADSVRGRFTISRDNSKNTLYLQMNSLRAEDTAVYYCARENIDAF
DVWG-
QGTLVTVSS
1B07-VL
[SEQ ID NO: 28]
QSVLTQPPSASGTPGQRVTISCSGSSSNIGNNAVNWYQQLPGTAPKLLIY
DNQQRP-
SGVPDRFSGSKSGTSASLAISGLRSEDEADYYCEAWDDRLFGPVFGGGTK
LTVLG
CDR regions
CDRH1:
[SEQ ID NO: 57]
SYGMH
CDRH2:
[SEQ ID NO: 58]
FTRYDGSNKYYADSVRG
CDRH3:
[SEQ ID NO: 59]
ENIDAFDV
CDRL1:
[SEQ ID NO: 60]
SGSSSNIGNNAVN
CDRL2:
[SEQ ID NO: 61]
DNQQRPS
CDRL3:
[SEQ ID NO: 62]
WDDRLFGPV
Antibody clone: 1C04
1C04-VH
[SEQ ID NO: 5]
EVQLLESGGGLVQPGGSLRLSCAASGFTFSSYAMSWVRQAPGKGLEWVS-
SISDSGAGRYYADSVEGRFTISRDNSKNTLYLQMNSLRAEDTAVYYCA-
RTHDSGELLDAFDIWGQGTLVTVSS
1C04-VL
[SEQ ID NO: 29]
QSVLTQPPSASGTPGQRVTISCSGSSSNIGSNHVLWYQQLPGTAPKLLIY
GNSNRPSG-
VPDRFSGSKSGTSASLAISGLRSEDEADYYCAAWDDSLNGWVFGGGTKLT
VLG
CDR regions
CDRH1:
[SEQ ID NO: 63]
SYAMS
CDRH2:
[SEQ ID NO: 64]
SISDSGAGRYYADSVEG
CDRH3:
[SEQ ID NO: 65]
THDSGELLDAFDI
CDRL1:
[SEQ ID NO: 66]
SGSSSNIGSNHVL
CDRL2:
[SEQ ID NO: 67]
GNSNRPS
CDRL3:
[SEQ ID NO: 68]
AAWDDSLNGWV
Antibody clone: 1E05
1E05-VH
[SEQ ID NO: 6]
EVQLLESGGGLVQPGGSLRLSCAASGFTFSTYAMNWVRQVPGKGLEWVAV
ISYD-
GSNKNYVDSVKGRFTISRDNSKNTLYLQMNSLRAEDTAVYYCARNFDNSG
YAIPDAFDIWGQGTLVTVSS
1E05-VL
[SEQ ID NO: 30]
QSVLTQPPSASGTPGQRVTISCTGSSSNIGAGYDVHWYQQLPGTAPKLL
I-
YDNNSRPSGVP-
DRFSGSKSGTSASLAISGLRSEDEADYYCAAWDDSLGGPVFGGGTKLTVL
G
CDR regions
CDRH1:
[SEQ ID NO: 69]
TYAMN
CDRH2:
[SEQ ID NO: 70]
VISYDGSNKNYVDSVKG
CDRH3:
[SEQ ID NO: 71]
NFDNSGYAIPDAFDI
CDRL1:
[SEQ ID NO: 72]
TGSSSNIGAGYDVH
CDRL2:
[SEQ ID NO: 73]
DNNSRPS
CDRL3:
[SEQ ID NO: 74]
AAWDDSLGGPV
Antibody clone: 2A09
2A09-VH
[SEQ ID NO: 7]
EVQLLESGGGLVQPGGSLRLSCAASGFTFSNAWMSWVR-
QAPGKGLEWVAYISRDADITHY-
PASVKGRFTISRDNSKNTLYLQMNSLRAEDTAVYYCTTGFDYAGDDAFDI
WGQGTLVTVSS
2A09-VL
[SEQ ID NO: 31]
QSVLTQPPSASGTPGQRVTISCSGSSSNIGSNAVNWYQQLPGTAPKLLI-
YGNSDRPSGVP-
DRFSGSKSGTSASLAISGLRSEDEADYYCAAWDDSLNGRWVFGGGTKLTV
LG
CDR regions
CDRH1:
[SEQ ID NO: 75]
NAWMS
CDRH2:
[SEQ ID NO: 76]
YISRDADITHYPASVKG
CDRH3:
[SEQ ID NO: 77]
GFDYAGDDAFDI
CDRL1:
[SEQ ID NO: 78]
SGSSSNIGSNAVN
CDRL2:
[SEQ ID NO: 79]
GNSDRPS
CDRL3:
[SEQ ID NO: 80]
AAWDDSLNGRWV
Antibody clone: 2B08
2B08-VH
[SEQ ID NO: 8]
EVQLLESGGGLVQPGGSLRLSCAASGFTFSDYYMSWVR-
QAPGKGLEWVALIGHDGNN-
KYYLDSLEGRFTISRDNSKNTLYLQMNSLRAEDTAVYYCARATDSGYDLL
YWGQGTLVTVSS
2B08-VL
[SEQ ID NO: 32]
QSVLTQPPSASGTPGQRVTISCSGSSSNIGNNAVNWYQQLPGTAP-
KLLIYYDDLLPSGVP-
DRFSGSKSGTSASLAISGLRSEDEADYYCTTWDDSLSGVVFGGGTKLTVL
G
CDR regions
CDRH1:
[SEQ ID NO: 81]
DYYMS
CDRH2:
[SEQ ID NO: 82]
LIGHDGNNKYYLDSLEG
CDRH3:
[SEQ ID NO: 83]
ATDSGYDLLY
CDRL1:
[SEQ ID NO: 84]
SGSSSNIGNNAVN
CDRL2:
[SEQ ID NO: 85]
YDDLLPS
CDRL3:
[SEQ ID NO: 86]
TTWDDSLSGVV
Antibody clone: 2E8-VH
2E8-VH
[SEQ ID NO: 9]
EVQLLESGGGLVQPGGSLRLS-
CAASGFTFSDYYMSWIRQAPGKGLEWVSAIGFSDDNTY-
YADSVKGRFTISRDNSKNTLYLQMNSLRAEDTAVYYCAGGDGSGWSFWGQ
GTLVTVSS
2E8-VL
[SEQ ID NO: 33]
QSVLTQPPSASGTPGQRVTISCSGSSSNIGNNAVNWYQQLPGTAPKLLIY
DNN-
KRPSGVP-
DRFSGSKSGTSASLAISGLRSEDEADYYCATWDDSLRGWVFGGGTKLTVL
G
CDR regions
CDRH1:
[SEQ ID NO: 87]
DYYMS
CDRH2:
[SEQ ID NO: 88]
AIGFSDDNTYYADSVKG
CDRH3:
[SEQ ID NO: 89]
GDGSGWSF
CDRL1:
[SEQ ID NO: 90]
SGSSSNIGNNAVN
CDRL2:
[SEQ ID NO: 91]
DNNKRPS
CDRL3:
[SEQ ID NO: 92]
ATWDDSLRGWV
Antibody clone: 5C04
5C04-VH
[SEQ ID NO: 10]
EVQLLESGGGLVQPGGSLRLSCAASGFTFSNYGMHWVRQAPGKGLEWVAV
ISYDGS-
NKYYADSVKGRFTISRDNSKNTLYLQMNSLRAEDTAVYYCAREWRDAFDI
WGQ-
GTLVTVSS
5C04-VL
[SEQ ID NO: 34]
QSVLTQPPSASGTPGQRVTISCTGSSSNIGAGYDVHWYQQLPGTAPKLLI
YSD-
NQRPSGVPDRFSGSKSGTSASLAISGLRSEDEADYYCAAWDDSLSGSWV
F-
GGGTKLTVLG
CDR regions
CDRH1:
[SEQ ID NO: 93]
NYGMH
CDRH2:
[SEQ ID NO: 94]
VISYDGSNKYYADSVKG
CDRH3:
[SEQ ID NO: 95]
WRDAFDI
CDRL1:
[SEQ ID NO: 96]
TGSSSNIGAGYDVH
CDRL2:
[SEQ ID NO: 97]
SDNQRPS
CDRL3:
[SEQ ID NO: 98]
AAWDDSLSGSWV
Antibody clone: 5C05
5C05-VH
[SEQ ID NO: 11]
EVQLLESGGGLVQPGGSLRLSCAASGFTFSTYGMHWVRQAPGKGLEWVAV
ISYD-
GSNKY-
YADSVKGRFTISRDNSKNTLYLQMNSLRAEDTAVYYCARENFDAFDVWGQ
GTLVTVSS
5C05-VL
[SEQ ID NO: 35]
QSVLTQPPSASGTPGQRVTISCTGSSSNIGAGYDVHWYQQLPGTAPKLLI
YSNS-
QRPSGVP-
DRFSGSKSGTSASLAISGLRSEDEADYYCAAWDDSLNGQVVFGGGTKLTV
LG
CDR regions
CDRH1:
[SEQ ID NO: 99]
TYGMH
CDRH2:
[SEQ ID NO: 100]
VISYDGSNKYYADSVKG
CDRH3:
[SEQ ID NO: 101]
ENFDAFDV
CDRL1:
[SEQ ID NO: 102]
TGSSSNIGAGYDVH
CDRL2:
[SEQ ID NO: 103]
SNSQRPS
CDRL3:
[SEQ ID NO: 104]
AAWDDSLNGQVV
Antibody clone: 5D07
5D07-VH
[SEQ ID NO: 12]
EVQLLESGGGLVQPGGSLRLSCAASGFTFSTYGMHWVR-
QAPGKGLEWVAVIAYDGSKKDY-
ADSVKGRFTISRDNSKNTLYLQMNSLRAEDTAVYYCAREYRDAFDIWGQG
TLVTVSS
5D07-VL
[SEQ ID NO: 36]
QSVLTQPPSASGTPGQRVTISCTGSSSNIGAGYDVHWYQQLPGTAPKLL
I-
YGNSNRPSGVP-
DRFSGSKSGTTASLAISGLRSEDEADYYCAAWDDSVSGWMFGGGTKLTVL
G
CDR regions
CDRH1:
[SEQ ID NO: 105]
TYGMH
CDRH2:
[SEQ ID NO: 106]
VIAYDGSKKDYADSVKG
CDRH3:
[SEQ ID NO: 107]
EYRDAFDI
CDRL1:
[SEQ ID NO: 108]
TGSSSNIGAGYDVH
CDRL2:
[SEQ ID NO: 109]
GNSNRPS
CDRL3:
[SEQ ID NO: 110]
AAWDDSVSGWM
Antibody clone: 5E12
5E12-VH
[SEQ ID NO: 13]
EVQLLESGGGLVQPGGSLRLSCAASGFTFSSYGMHWVRQAPGKGLEWVAV
ISYDGIN-
KDYADSMKGRFTISRDNSKNTLYLQMNSLRAEDTAVYYCARERKDAFDIW
GQGT-
LVTVSS
5E12-VL
[SEQ ID NO: 37]
QSVLTQPPSASGTPGQRVTISCTGSSSNIGAGYDVHWYQQLPGTAPKLLI
YSNNQR-
PSGVPDRFSGSKSGTSASLAISGLRSEDEADYYCATWDDSLNGLVFGGGT
KLTVLG
CDR regions
CDRH1:
[SEQ ID NO: 111]
SYGMH
CDRH2:
[SEQ ID NO: 112]
VISYDGINKDYADSMKG
CDRH3:
[SEQ ID NO: 113]
ERKDAFDI
CDRL1:
[SEQ ID NO: 114]
TGSSSNIGAGYDVH
CDRL2:
[SEQ ID NO: 115]
SNNQRPS
CDRL3:
[SEQ ID NO: 116]
ATWDDSLNGLV
Antibody clone: 5G08
5G08-VH
[SEQ ID NO: 14]
EVQLLESGGGLVQPGGSLRLSCAASGFTFNNYGMHWVRQAPGKGLEWVAV
ISYD-
GSNRYYADSVKGRFTMSRDNSKNTLYLQMNSLRAEDTAVYYCARDRWNGM
DV-
WGQGTLVTVSS
5G08-VL
[SEQ ID NO: 38]
QSVLTQPPSASGTPGQRVTISCSGSSSNIGAGYDVHWYQQLPGTAPKLLI
YANNQRP-
SGVPDRFSGSKSGTSASLAISGLRSEDEADYYCAAWDDSLNGPWVFGGGT
KLTVLG
CDR regions
CDRH1:
[SEQ ID NO: 117]
NYGMH
CDRH2:
[SEQ ID NO: 118]
VISYDGSNRYYADSVKG
CDRH3:
[SEQ ID NO: 119]
DRWNGMDV
CDRL1:
[SEQ ID NO: 120]
SGSSSNIGAGYDVH
CDRL2:
[SEQ ID NO: 121]
ANNQRPS
CDRL3:
[SEQ ID NO: 122]
AAWDDSLNGPWV
Antibody clone: 5H06
5H06-VH
[SEQ ID NO: 15]
EVQLLESGGGLVQPGGSLRLSCAASGFTFSSYGMHWVRQAPGKGLEWVAV
ISYDGS-
DTAYADSVKGRFTISRDNSKNTLYLQMNSLRAEDTAVYYCARDHSVIGAF
DIWGQ-
GTLVTVSS
5H06-VL
[SEQ ID NO: 39]
QSVLTQPPSASGTPGQRVTISCSGSSSNIGSNTVNWYQQLPGTAPKLLIY
DNNKRP-
SGVPDRFSGSKSGTSASLAISGLRSEDEADYYCSSYAGSNNVVFGGGTKL
TVLG
CDR regions
CDRH1:
[SEQ ID NO: 123]
SYGMH
CDRH2:
[SEQ ID NO: 124]
VISYDGSDTAYADSVKG
CDRH3:
[SEQ ID NO: 125]
DHSVIGAFDI
CDRL1:
[SEQ ID NO: 126]
SGSSSNIGSNTVN
CDRL2:
[SEQ ID NO: 127]
DNNKRPS
CDRL3:
[SEQ ID NO: 128]
SSYAGSNNVV
Antibody clone: 6A09
6A09-VH
[SEQ ID NO: 16]
EVQLLESGGGLVQPGGSLRLSCAASGFTFSSYGMHWVRQAPGKGLEWVAV
TSYDGN-
TKYYANSVKGRFTISRDNSKNTLYLQMNSLRAEDTAVYYCAREDCGGDCF
DYW-
GQGTLVTVSS
6A09-VL
[SEQ ID NO: 40]
QSVLTQPPSASGTPGQRVTISCTGSSSNIGAGYDVHWYQQLPGTAPKLLI
YGNSNRPS-
GVPDRFSGSKSGTSASLAISGLRSEDEADYYCAAWDDSLNEGVFGGGTKL
TVLG
CDR regions
CDRH1:
[SEQ ID NO: 129]
SYGMH
CDRH2:
[SEQ ID NO: 130]
VTSYDGNTKYYANSVKG
CDRH3:
[SEQ ID NO: 131]
EDCGGDCFDY
CDRL1:
[SEQ ID NO: 132]
TGSSSNIGAGYDVH
CDRL2:
[SEQ ID NO: 133]
GNSNRPS
CDRL3:
[SEQ ID NO: 134]
AAWDDSLNEGV
Antibody clone: 6B01
6B01-VH
[SEQ ID NO: 17]
EVQLLESGGGLVQPGGSLRLSCAASGFTFSNYGMHWVRQAPGKGLEWVAV
ISYDGS-
NKYYADSVKGRFTISRDNSKNTLYLQMNSLRAEDTAVYYCARDQLGEAFD
IWGQGT-
LVTVSS
6B01-VL
[SEQ ID NO: 41]
QSVLTQPPSASGTPGQRVTISCTGSSSNIGAGYDVHWYQQLPGTAPKLLI
YDNNKRPS-
GVPDRFSGSKSGTSASLAISGLRSEDEADYYCATWDDSLSGPVFGGGTKL
TVLG
CDR regions
CDRH1:
[SEQ ID NO: 135]
NYGMH
CDRH2:
[SEQ ID NO: 136]
VISYDGSNKYYADSVKG
CDRH3:
[SEQ ID NO: 137]
DQLGEAFDI
CDRL1:
[SEQ ID NO: 138]
TGSSSNIGAGYDVH
CDRL2:
[SEQ ID NO: 139]
DNNKRPS
CDRL3:
[SEQ ID NO: 140]
ATWDDSLSGPV
Antibody clone: 6C11
6C11-VH
[SEQ ID NO: 18]
EVQLLESGGGLVQPGGSLRLSCAASGFTFDDYGMSWVRQAPGKGLEWVSA
ISGSG-
SSTYYADSVKGRFTISRDNSKNTLYLQMNSLRAEDTAVYYCAGGDIDYFD
YWGQGTL-
sVTVSS
6C11-VL
[SEQ ID NO: 42]
QSVLTQPPSASGTPGQRVTISCTGSSSNFGAGYDVHWYQQLPGTAPKLLI
YENNKRP-
SGVPDRFSGSKSGTSASLAISGLRSEDEADYYCAAWDDSLNGPVFGGGTK
LTVLG
CDR regions
CDRH1:
[SEQ ID NO: 141]
DYGMS
CDRH2:
[SEQ ID NO: 142]
AISGSGSSTYYADSVKG
CDRH3:
[SEQ ID NO: 143]
GDIDYFDY
CDRL1:
[SEQ ID NO: 144]
TGSSSNFGAGYDVH
CDRL2:
[SEQ ID NO: 145]
ENNKRPS
CDRL3:
[SEQ ID NO: 146]
AAWDDSLNGPV
Antibody clone: 6C12
6C12-VH
[SEQ ID NO: 19]
EVQLLESGGGLVQPGGSLRLSCAASGFTFSSYGMHWVRQAPGKGLEWVAV
ISYDGS-
NKYYADSVKGRFTISRDNSKNTLYLQMNSLRAEDTAVYYCARERRDAFDI
WGQGT-
LVTVSS
6C12-VL
[SEQ ID NO: 43]
QSVLTQPPSASGTPGQRVTISCTGSSSNIGAGYDVHWYQQLPGTAPKLLI
YSDNQ-
RPSGVPDRFSGSKSGTSASLAISGLRSEDEADYYCATWDSDTPVFGGGTK
LTVLG
CDR regions
CDRH1:
[SEQ ID NO: 147]
SYGMH
CDRH2:
[SEQ ID NO: 148]
VISYDGSNKYYADSVKG
CDRH3:
[SEQ ID NO: 149]
ERRDAFDI
CDRL1:
[SEQ ID NO: 150]
TGSSSNIGAGYDVH
CDRL2:
[SEQ ID NO: 151]
SDNQRPS
CDRL3:
[SEQ ID NO: 152]
ATWDSDTPV
Antibody clone: 6D01
6D01-VH
[SEQ ID NO: 20]
EVQLLESGGGLVQPGGSLRLSCAASGFTFSSYGMHWVRQAPGKGLEEVAV
ISYDGS-
NKYYADSVKGRFTISRDNSKNTLYLQMNSLRAEDTAMYYCARDHSAAGYF
DYWGQ-
GTLVTVSS
6D01-VL
[SEQ ID NO: 44]
QSVLTQPPSASGTPGQRVTISCSGSSSNIGSNTVNWYQQLPGTAPKLLIY
GNSIRPSG-
GPDRFSGSKSGTSASLAISGLRSEDEADYYCASWDDSLSSPVFGGGTKLT
VLG
CDR regions
CDRH1:
[SEQ ID NO: 153]
SYGMH
CDRH2:
[SEQ ID NO: 154]
VISYDGSNKYYADSVKG
CDRH3:
[SEQ ID NO: 155]
DHSAAGYFDY
CDRL1:
[SEQ ID NO: 156]
SGSSSNIGSNTVN
CDRL2:
[SEQ ID NO: 157]
GNSIRPS
CDRL3:
[SEQ ID NO: 158]
ASWDDSLSSPV
Antibody clone: 6G03
6G03-VH
[SEQ ID NO: 21]
EVQLLESGGGLVQPGGSLRLSCAASGFTFGSYGMHWVRQAPGKGLEWVSG
ISWDS-
AIIDYAGSVKGRFTISRDNSKNTLYLQMNSLRAEDTAVYYCAKDEAAAGA
FDIWGQG-
TLVTVSS
6G03-VL
[SEQ ID NO: 45]
QSVLTQPPSASGTPGQRVTISCTGSSSNIGAGYDVHWYQQLPGTAPKLLI
YGNTDRPS-
GVPDRFSGSKSGTSASLAISGLRSEDEADYYCAAWDDSLSGPVVFGGGTK
LTVLG
CDR regions
CDRH1:
[SEQ ID NO: 159]
SYGMH
CDRH2:
[SEQ ID NO: 160]
GISWDSAIIDYAGSVKG
CDRH3:
[SEQ ID NO: 161]
DEAAAGAFDI
CDRL1:
[SEQ ID NO: 162]
TGSSSNIGAGYDVH
CDRL2:
[SEQ ID NO: 163]
GNTDRPS
CDRL3:
[SEQ ID NO: 164]
AAWDDSLSGPVV
Antibody clone: 6G08
6G08-VH
[SEQ ID NO: 22]
EVQLLESGGGLVQPGGSLRLSCAASGFTLSSYGISWVRQAPGKGLEWVSG
ISGSGGN-
TYYADSVKGRFTISRDNSKNTLYLQMNSLRAEDTAVYYCASSVGAYANDA
FDIWGQ-
GTLVTVSS
6G08-VL
[SEQ ID NO: 46]
QSVLTQPPSASGTPGQRVTISCTGSSSNIGAGYDVHWYQQLPGTAPKLLI
YGDTNRPS-
GVPDRFSGSKSGTSASLAISGLRSEDEADYYCAAWDDSLNGPVFGGGTKL
TVLG
CDR regions
CDRH1:
[SEQ ID NO: 165]
SYGIS
CDRH2:
[SEQ ID NO: 166]
GISGSGGNTYYADSVKG
CDRH3:
[SEQ ID NO: 167]
SVGAYANDAFDI
CDRL1:
[SEQ ID NO: 168]
TGSSSNIGAGYDVH
CDRL2:
[SEQ ID NO: 169]
GDTNRPS
CDRL3:
[SEQ ID NO: 170]
AAWDDSLNGPV
Antibody clone: 6G11
6G11-VH
[SEQ ID NO: 23]
EVQLLESGGGLVQPGGSLRLSCAASGFTFSSYGMHWVRQAPGKGLEWMAV
ISYDGS-
NKYYADSVKGRFTISRDNSKNTLYLQMNSLRAEDTAVYYCARELYDAFDI
WGQGTL-
VTVSS
6G11-VL
[SEQ ID NO: 47]
QSVLTQPPSASGTPGQRVTISCTGSSSNIGAGYDVHWYQQLPGTAPKLLI
YADDHRP-
SGVPDRFSGSKSGTSASLAISGLRSEDEADYYCASWDDSQRAVIFGGGTK
LTVLG
CDR regions
CDRH1:
[SEQ ID NO: 171]
SYGMH
CDRH2:
[SEQ ID NO: 172]
VISYDGSNKYYADSVKG
CDRH3:
[SEQ ID NO: 173]
ELYDAFDI
CDRL1:
[SEQ ID NO: 174]
TGSSSNIGAGYDVH
CDRL2:
[SEQ ID NO: 175]
ADDHRPS
CDRL3:
[SEQ ID NO: 176]
ASWDDSQRAVI
Antibody clone: 6H08
6H08-VH
[SEQ ID NO: 24]
EVQLLESGGGLVQPGGSLRLSCAASGFTFNNYGMHWVRQAPGKGLEWVAV
ISYDGS-
NKYYADSVKGRFTISKDNSKNTLYLQMNSLRAEDTAVYYCAREYKDAFDI
WGQGTL-
VTVSS
6H08-VL
[SEQ ID NO: 48]
QSVLTQPPSASGTPGQRVTISCTGSSSNIGSNTVNWYQQLPGTAPKLLIY
DNNKRPS-
GVPDRFSGSKSGTSASLAISGLRSEDEADYYCQAWGTGIRVFGGGTKLTV
LG
CDR regions
CDRH1:
[SEQ ID NO: 177]
NYGMH
CDRH2:
[SEQ ID NO: 178]
VISYDGSNKYYAD SVKG
CDRH3:
[SEQ ID NO: 179]
EYKDAFDI
CDRL1:
[SEQ ID NO: 180]
TGSSSNIGSNTVN
CDRL2:
[SEQ ID NO: 181]
DNNKRPS
CDRL3:
[SEQ ID NO: 182]
QAWGTGIRV
Antibody clone: 7C07
7C07-VH
[SEQ ID NO: 25]
EVQLLESGGGLVQPGGSLRLSCAASGFTFSSYGMHWVRQAPGKGLEWVAV
ISYDGS-
NKYYADSVKGRFTISRDNSQNTLYLQMNSLRAEDTAVYYCAREFGYIILD
YWGQG-
TLVTVSS
7C07-VL
[SEQ ID NO: 49]
QSVLTQPPSASGTPGQRVTISCSGSSSNIGSNTVNWYQQLPGTAPKKKIY
RDYER-
PSGVPDRFSGSKSGTSASLAISGLRSEDEADYYCMAWDDSLSGVVFGGGT
KLTVLG
CDR regions
CDRH1:
[SEQ ID NO: 183]
SYGMH
CDRH2:
[SEQ ID NO: 184]
VISYDGSNKYYADSVKG
CDRH3:
[SEQ ID NO: 185]
EFGYIILDY
CDRL1:
[SEQ ID NO: 186]
SGSSSNIGSNTVN
CDRL2:
[SEQ ID NO: 187]
RDYERPS
CDRL3:
[SEQ ID NO: 188]
MAWDDSLSGVV
Antibody clone: 4B02
4B02-VH
[SEQ ID NO: 26]
EVQLLESGGGLVQPGGSLRLSCAASGFTFSNHGMHWVRQAPGKGLEWVAV
ISYDGT-
NKYYADSVRGRFTISRDNSKNTLYLQMNSLRAEDTAVYYCARETWDAFDV
WGQGTLV-
TVSS
4B02-VL
[SEQ ID NO: 50]
QSVLTQPPSASGTPGQRVTISCSGSSSNIGSNNANWYQQLPGTAPKLLIY
DNN-
KRPSGVPDRFSGSKSGTSASLAISGLRSEDEADYYCQAWDSSTVVFGGGT
KLTVLG
CDR regions
CDRH1:
[SEQ ID NO: 189]
NHGMH
CDRH2:
[SEQ ID NO: 190]
VISYDGTNKYYADSVRG
CDRH3:
[SEQ ID NO: 191]
ETWDAFDV
CDRL1:
[SEQ ID NO: 192]
SGSSSNIGSNNAN
CDRL2:
[SEQ ID NO: 193]
DNNKRPS
CDRL3:
[SEQ ID NO: 194]
QAWDSSTVV

In some embodiments, which are sometimes preferred embodiments, the antibody molecule that specifically binds FcγRIIb comprises the following CDR regions: SEQ ID NO: 171 (CDRH1), SEQ ID NO: 172 (CDRH2), SEQ ID NO: 173 (CDRH3), SEQ ID NO: 174 (CDRL1), SEQ ID NO: 175 (CDRL2) and SEQ ID NO: 176 (CDRL3), i.e. the CDR regions of clone 6G11.

In some embodiments, which are sometimes preferred embodiments, the antibody molecule that specifically binds FcγRIIb comprises the following constant regions: SEQ ID NO: 1 (CH) and SEQ ID NO: 2 (CL) and the following variable regions: SEQ ID NO: 23 (VL) and SEQ ID NO: 47 (VH) i.e. the constant and variable regions of clone 6G11.

In some embodiments, the anti-PD-1 antibody molecule is a human antibody molecule or an antibody molecule of human origin. In some such embodiments, the human antibody molecule or antibody molecule of human origin is an IgG antibody. In some such embodiments the human antibody molecule or antibody molecule of human origin is an IgG4.

The anti-PD-1 antibody molecule is an antibody molecule that binds specifically to PD-1.

In some embodiments, the anti-PD-1 antibody molecule blocks binding of PD-L1 and/or PD-L2 to PD-1, and may then be regarded as a PD-1 antagonist.

In some embodiments, the anti-PD-1 antibody molecule is a humanized antibody molecule.

In some embodiments the anti-PD-1 antibody molecule is a chimeric antibody.

As mentioned above, the anti-PD-1 antibody must have the ability to engage FcγRs.

In some embodiments, the anti-PD-1 antibody molecule is selected from the group consisting of nivolumab (OPDIVO®), pembrolizumab (KEYTRUDA®) and cemiplimab (LIBTAYO®).

In some embodiments the antibody molecule that specifically binds FcγRIIb and the anti-PD-1 antibody molecule are administered simultaneously to the patient, meaning that they are either administered together at one or separately very close in time to each other.

In some embodiments the antibody molecule that specifically binds FcγRIIb is administered to the patient prior to administration of the anti-PD-1 antibody molecule. Such sequential administration may be achieved by temporal separation of the two antibodies. Alternatively, or in combination with the first option, the sequential administration may also be achieved by spatial separation of the two antibody molecules, by administration of the antibody molecule that specifically binds FcγRIIb in a way, such as intratumoral, so that it reaches the cancer prior to the anti-PD-1 antibody molecule, which is then administered in a way, such as systemically, so that it reaches the cancer after the antibody molecule that specifically binds FcγRIIb.

In some embodiments the anti-PD-1 antibody molecule is administered to the patient prior to administration of the antibody molecule that specifically binds FcγRIIb. Similarly to what is described above, such sequential administration may be achieved by temporal separation of the two antibodies and/or by spatial separation of the two antibody molecules. For spatial administration, the anti-PD-1 antibody molecule is administered in a way, such as intratumoral, so that it reaches the cancer prior to the antibody molecule that specifically binds FcγRIIb, which is then administered in a way, such as systemically, so that it reaches the cancer after the PD-1 antibody molecule.

It would be known to the person skilled in medicine, that medicines can be modified with different additives, for example to change the rate in which the medicine is absorbed by the body; and can be modified in different forms, for example to allow for a particular administration route to the body.

Accordingly, we include that the composition, and/or antibody, and/or medicament of the invention may be combined with an excipient and/or a pharmaceutically acceptable carrier and/or a pharmaceutically acceptable diluent and/or an adjuvant.

We also include that the composition, and/or antibody, and/or medicament of the invention may be suitable for parenteral administration including aqueous and/or non-aqueous sterile injection solutions which may contain anti-oxidants, and/or buffers, and/or bacteriostats, and/or solutes which render the formulation isotonic with the blood of the intended recipient; and/or aqueous and/or non-aqueous sterile suspensions which may include suspending agents and/or thickening agents. The composition, and/or antibody, and/or agent, and/or medicament of the invention may be presented in unit-dose or multi-dose containers, for example sealed ampoules and vials, and may be stored in a freeze-dried (i.e. lyophilized) condition requiring only the addition of the sterile liquid carrier, for example water for injections, immediately prior to use.

Extemporaneous injection solutions and suspensions may be prepared from sterile powders, and/or granules, and/or tablets of the kind previously described.

For parenteral administration to human patients, the daily dosage level of the antibody molecule that specifically binds FcγRIIb and/or the anti-PD-1 antibody molecule will usually be from 1 mg/kg bodyweight of the patient to 20 mg/kg, or in some cases even up to 100 mg/kg administered in single or divided doses. Lower doses may be used in special circumstances, for example in combination with prolonged administration. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited, and such are within the scope of this invention.

Typically, the composition and/or medicament of the invention will contain the antibody molecule that specifically binds FcγRIIb and/or the anti-PD-1 antibody at a concentration of between approximately 2 mg/ml and 150 mg/ml or between approximately 2 mg/ml and 200 mg/ml. In a preferred embodiment, the medicaments and/or compositions of the invention will contain the antibody molecule that specifically binds FcγRIIb and/or the anti-PD-1 antibody molecule at a concentration of 10 mg/ml.

Generally, in humans, oral or parenteral administration of the composition, and/or antibody, and/or agent, and/or medicament of the invention is the preferred route, being the most convenient. For veterinary use, the composition, and/or antibody, and/or agent and/or medicament of the invention are administered as a suitably acceptable formulation in accordance with normal veterinary practice and the veterinary surgeon will determine the dosing regimen and route of administration which will be most appropriate for a particular animal. Thus, the present invention provides a pharmaceutical formulation comprising an amount of an antibody and/or agent of the invention effective to treat various conditions (as described above and further below). Preferably, the composition, and/or antibody, and/or agent, and/or medicament is adapted for delivery by a route selected from the group comprising: intravenous (IV); subcutaneous (SC), intramuscular (IM), or intratumoral.

The present invention also includes composition, and/or antibody, and/or agent, and/or medicament comprising pharmaceutically acceptable acid or base addition salts of the polypeptide binding moieties of the present invention. The acids which are used to prepare the pharmaceutically acceptable acid addition salts of the aforementioned base compounds useful in this invention are those which form non-toxic acid addition salts, i.e. salts containing pharmacologically acceptable anions, such as the hydrochloride, hydrobromide, hydroiodide, nitrate, sulphate, bisulphate, phosphate, acid phosphate, acetate, lactate, citrate, acid citrate, tartrate, bitartrate, succinate, maleate, fumarate, gluconate, saccharate, benzoate, methanesulphonate, ethanesulphonate, benzenesulphonate, p-toluenesulphonate and pamoate [i.e. 1,1-methylene-bis-(2-hydroxy-3 naphthoate)] salts, among others. Pharmaceutically acceptable base addition salts may also be used to produce pharmaceutically acceptable salt forms of the agents according to the present invention. The chemical bases that may be used as reagents to prepare pharmaceutically acceptable base salts of the present agents that are acidic in nature are those that form non-toxic base salts with such compounds. Such non-toxic base salts include, but are not limited to those derived from such pharmacologically acceptable cations such as alkali metal cations (e.g. potassium and sodium) and alkaline earth metal cations (e.g. calcium and magnesium), ammonium or water-soluble amine addition salts such as N-methylglucamine-(meglumine), and the lower alkanolammonium and other base salts of pharmaceutically acceptable organic amines, among others. The agents and/or polypeptide binding moieties of the invention may be lyophilised for storage and reconstituted in a suitable carrier prior to use. Any suitable lyophilisation method (e.g. spray drying, cake drying) and/or reconstitution techniques can be employed. It will be appreciated by those skilled in the art that lyophilisation and reconstitution can lead to varying degrees of antibody activity loss (e.g. with conventional immunoglobulins, IgM antibodies tend to have greater activity loss than IgG antibodies) and that use levels may have to be adjusted upward to compensate. In one embodiment, the lyophilised (freeze dried) polypeptide binding moiety loses no more than about 20%, or no more than about 25%, or no more than about 30%, or no more than about 35%, or no more than about 40%, or no more than about 45%, or no more than about 50% of its activity (prior to lyophilisation) when re-hydrated.

The combination of an antibody molecule that specifically binds FcγRIIb and an anti-PD-1 antibody molecule can be used use in the treatment of cancer.

“Patient” as the term is used herein refers to an animal, including human, that has been diagnosed as having an FcγRIIb negative cancer or as having a cancer that is considered as likely to be FcγRIIb negative cancer and/or that exhibits symptoms of such a cancer.

We include that the patient could be mammalian or non-mammalian. Preferably, the patient is a human or is a mammalian, such as a horse, or a cow, or a sheep, or a pig, or a camel, or a dog, or a cat. Most preferably, the mammalian patient is a human.

By “exhibit”, we include that the subject displays a cancer symptom and/or a cancer diagnostic marker, and/or the cancer symptom and/or a cancer diagnostic marker can be measured, and/or assessed, and/or quantified.

It would be readily apparent to the person skilled in medicine what the cancer symptoms and cancer diagnostic markers would be and how to measure and/or assess and/or quantify whether there is a reduction or increase in the severity of the cancer symptoms, or a reduction or increase in the cancer diagnostic markers; as well as how those cancer symptoms and/or cancer diagnostic markers could be used to form a prognosis for the cancer.

Cancer treatments are often administered as a course of treatment, which is to say that the therapeutic agent is administered over a period of time. The length of time of the course of treatment will depend on a number of factors, which could include the type of therapeutic agent being administered, the type of cancer being treated, the severity of the cancer being treated, and the age and health of the patient, amongst others reasons.

By “during the treatment”, we include that the patient is currently receiving a course of treatment, and/or receiving a therapeutic agent, and/or receiving a course of a therapeutic agent.

The patient to be treated in accordance with the present invention has a cancer characterized by PD-1 positive tumors.

In some embodiments the cancer to be treated is a solid cancer.

In some embodiments the solid cancer to be treated is a cancer for which the treatment normally consists of or comprises immunotherapy with an anti-PD-1 antibody.

In some embodiments, the cancer to be treated is selected from the group consisting of melanoma; lung cancer, including small cell lung cancer (SCLC) and non-small cell lung carcinoma (NSCLC) (including non-squamous NSCLC and squamous NSCLC, and including metastatic NSCLC); head and neck cancer, including head and neck squamous cell carcinoma (HNSCC); Hodgkin lymphoma; primary mediastinal B-cell lymphoma (PMBCL); bladder cancer, including advanced urothelial carcinoma; colorectal cancer, including cancer that is instability-high (MSI-H) and/or mismatch repair deficient (dMMR); gastric cancer, including advanced gastric cancer and gastric or gastroesophageal junction (GEJ) adenocarcinoma; cervical cancer; liver cancer, including hepatocellular carcinoma; Merkel cell carcinoma (MCC); kidney cancer, including renal cell carcinoma (RCC) and cutaneous squamous cell carcinoma (CSCC), including locally advanced CSCC in patients who are not candidates for curative surgery or curative radiation. It will be known to a person skilled in the art that the number and types of indications relevant to anti-PD-1 immunotherapy is rapidly expanding.

In some embodiments the cancer is refractory cancer. In some such embodiments the refractory cancer is a cancer that is found to be resistant to treatment with an anti-PD-1 antibody already at the beginning of treatment. This resistant can be evidenced either by the patient not responding at all to the treatment or by some progress of the cancer despite treatment. In some embodiments the refractory cancer is a cancer that becomes resistant to an anti-PD-1 antibody during treatment with that antibody, which means that the patient stops responding to the treatment or shows a decreased response to the treatment. In some embodiments the refractory cancer is resistant after, or at the final stages of, successful treatment with an anti-PD-1 antibody, which means that an anti-PD-1 antibody will have no or reduced effect if the cancer relapses.

Each one of the above described cancers is well-known, and the symptoms and cancer diagnostic markers are well described, as are the therapeutic agents used to treat those cancers. Accordingly, the symptoms, cancer diagnostic markers, and therapeutic agents used to treat the above mentioned cancer types would be known to those skilled in medicine.

Clinical definitions of the diagnosis, prognosis and progression of a large number of cancers rely on certain classifications known as staging. Those staging systems act to collate a number of different cancer diagnostic markers and cancer symptoms to provide a summary of the diagnosis, and/or prognosis, and/or progression of the cancer. It would be known to the person skilled in oncology how to assess the diagnosis, and/or prognosis, and/or progression of the cancer using a staging system, and which cancer diagnostic markers and cancer symptoms should be used to do so.

By “cancer staging”, we include the Rai staging, which includes stage 0, stage I, stage II, stage III and stage IV, and/or the Binet staging, which includes stage A, stage B and stage C, and/or the Ann Arbour staging, which includes stage I, stage II, stage III and stage IV.

It is known that cancer can cause abnormalities in the morphology of cells. These abnormalities often reproducibly occur in certain cancers, which means that examining these changes in morphology (otherwise known as histological examination) can be used in the diagnosis or prognosis of cancer. Techniques for visualizing samples to examine the morphology of cells, and preparing samples for visualization, are well known in the art; for example, light microscopy or confocal microscopy.

By “histological examination”, we include the presence of small, mature lymphocyte, and/or the presence of small, mature lymphocytes with a narrow border of cytoplasm, the presence of small, mature lymphocytes with a dense nucleus lacking discernible nucleoli, and/or the presence of small, mature lymphocytes with a narrow border of cytoplasm, and with a dense nucleus lacking discernible nucleoli, and/or the presence of atypical cells, and/or cleaved cells, and/or prolymphocytes.

It is well known that cancer is a result of mutations in the DNA of the cell, which can lead to the cell avoiding cell death or uncontrollably proliferating. Therefore, examining these mutations (also known as cytogenetic examination) can be a useful tool for assessing the diagnosis and/or prognosis of a cancer. An example of this is the deletion of the chromosomal location 13q14.1 which is characteristic of chronic lymphocytic leukaemia. Techniques for examining mutations in cells are well known in the art; for example, fluorescence in situ hybridization (FISH).

By “cytogenetic examination”, we include the examination of the DNA in a cell, and, in particular the chromosomes. Cytogenetic examination can be used to identify changes in DNA which may be associated with the presence of a refractory cancer and/or relapsed cancer. Such may include: deletions in the long arm of chromosome 13, and/or the deletion of chromosomal location 13q14.1, and/or trisomy of chromosome 12, and/or deletions in the long arm of chromosome 12, and/or deletions in the long arm of chromosome 11, and/or the deletion of 11q, and/or deletions in the long arm of chromosome 6, and/or the deletion of 6q, and/or deletions in the short arm of chromosome 17, and/or the deletion of 17p, and/or the t(11:14) translocation, and/or the (q13:q32) translocation, and/or antigen gene receptor rearrangements, and/or BCL2 rearrangements, and/or BCL6 rearrangements, and/or t(14:18) translocations, and/or t(11:14) translocations, and/or (q13:q32) translocations, and/or (3:v) translocations, and/or (8:14) translocations, and/or (8:v) translocations, and/or t(11:14) and (q13:q32) translocations.

It is known that patients with cancer exhibit certain physical symptoms, which are often as a result of the burden of the cancer on the body. Those symptoms often reoccur in the same cancer, and so can be characteristic of the diagnosis, and/or prognosis, and/or progression of the disease. A person skilled in medicine would understand which physical symptoms are associated with which cancers, and how assessing those physical systems can correlate to the diagnosis, and/or prognosis, and/or progression of the disease. By “physical symptoms”, we include hepatomegaly, and/or splenomegaly.

### BRIEF DESCRIPTION OF THE DRAWINGS

In the examples below, reference is made to the following figures:

FIG. 1 shows PD-1 expression on human Jurkat T cells transfected with PD-1. PD-1 transfected Jurkat cells were sorted into low, medium and high PD-1 expressing cells. Following expansion, the PD-1 expression was quantified in the three different subsets and the number of PD-1 molecules/cell is shown in FIG. 1A (low), FIG. 1B (medium) and FIG. 1C (high).

FIG. 2 shows that BI-1206 (6G11 WT) inhibits PD-1 mediated phagocytosis of medium and high, but not low, expressing cells. FIG. 2A illustrates an example showing the phagocytosed Jurkat cells. FL4 on the y-axis depicts CD14+ macrophages and FL1 on the x-axis depict CFSE labeled Jurkat cells. The encircled upper right quadrant therefore shows double positive CD14+ CFSE+ cells which are phagocytosed Jurkat cells. The example shows phagocytosis of PD-1 high expressing Jurkat cells. FIG. 2B shows phagocytosis of PD-1 mid-expressing Jurkat cells and FIG. 2C shows high expressing Jurkat cells. The values are normalized towards isotype opsonization (set to zero %) and anti CD3 opsonization (OKT3 hlgG1, set to 100%). The figure shows that BI-1206 (denoted 6G11 WT in the figure) inhibits nivolumab mediated phagocytosis at all concentrations tested. Furthermore, the figure shows that the 6G11 antibody need an intact Fc-part to inhibit phagocytosis, since disruption of FcγR binding caused by inducing a mutation in position 297 from amino acid asparagine (N) to amino acid glutamine (Q) (i.e. the antibody here denoted 6G11 NQ) diminishes it's capacity to inhibit nivolumab mediated phagocytosis. The figure shows 2 experiments for the mid-expressing cells and 3 experiments for the high expressing cells. FIG. 2D shows that there is no nivolumab mediated phagocytosis in the low-expressing cells.

FIG. 3 shows that Fc:FcγR-binding proficient anti-FcγRIIB (AT-130-2 mIgG2a and mIgG1), but not Fc:FcγR-binding impaired anti-FcγRIIB (AT-130-2 mIgG1 NA), enhances anti-PD-1 antibody therapeutic efficacy and survival in vivo. CT26 (Figures A and B) or MC38 (FIG. 3C and FIG. 3D) tumor-bearing mice were treated three times (days 8, 12 and 15 post inoculation of 5×10⁵ tumor cells S.C. in 100 μl PBS) with 200 μg of anti-PD-1 (Clone 29F.1A12; Bioxcell) antibody alone or in combination with 200 μg indicated anti-FcγRIIB antibody variant or isotype control (WR17). For the first treatment AT130-2 was administered 6 hours prior to anti-PD1 antibody. For subsequent treatments both antibodies were given together. All injections were I.P. in 2001 PBS. Tumors were considered terminal when they reached an area of 400 mm² for CT26 or 225 mm² for MC38Graphs show tumor growth (FIG. 3A and FIG. 3C) and survival (FIG. 3B and FIG. 3D) of animals. (**P<0.01; Log-Rank test). The experiments were done in female mice aged 8-14 weeks.

FIG. 4 shows PD-1 expression on immune cells in tumor-bearing mice. Immune cells from mice tumors were quantified for PD-1 expression. Mice were injected with MC38 cells and tumors were collected after ˜20 days. Cells were stained for different T cell subsets and PD-1 expression on CD8+ T cells were analyzed by FACS. Mean fluorescent intensity values of PD-1 on cells were correlated to values from Quantum™ Simply Cellular® beads, stained with the same anti PD-1 antibody, to determine the number of receptors per cell.

FIG. 5 shows Jurkat cells expressing different levels of PD-1, i.e. PD-1 low, PD-1 medium (mid) and PD-1 high. The PD-1 expressions were defined using saturating concentration of Alexa Fluor 647 human anti-human PD-1 (pembrolizumab).

FIG. 6 shows PD-1 expression on “Jurkat PD-1 mid cells”. The gate shows the full width/half height gate used to define the lower end of PD-1 “mid-high” expression on tumor samples.

FIG. 7 illustrates the gating strategy used to define the PD-1 expression on human tumor samples. First the CD45+ events were defined (A) followed by live cells (B), then CD3+(C) or CD3+CD8+(D) were defined. The PD-1 high gate were set in the CD3+(E) and CD3+CD8+ population respectively (F). The PD-1 “high” gate were defined based on the PD-1 transfected Jurkat cells and the lower end was set according to the low end of the full width/half height gate on the PD-1 mid Jurkat cells. (G) and (H) shows the FMO for Alexa Fluor 647 human anti-human PD-1 (pembrolizumab) in the CD3+ and CD3+CD8+ population respectively.

FIG. 8 shows a table summarizing data for each patient from which tumor samples were obtained, including patient characteristics including PD-1 expression and predicted response.

FIG. 9 illustrates percentage of PD-1 medium-high expressing CD3+ and CD3+CD8+ lymphocytes. The dotted line defines 10%. The letters (F, G, H etc) correspond to the Patient ID of the table in FIG. 8.

### EXAMPLES

Specific, non-limiting examples which embody certain aspects of the invention will now be described. These examples should be read together with the brief description of the drawings provided above.

### Example 1

### Transfection of Jurkat Cells

For transfection of Jurkat cells, the cells were cultured in RPMI-1640 medium containing 10% fetal calf serum, FCS (Sigma), L-glutamine (Life Technologies), sodium pyruvate (Life Technologies) and Pen-Strep (Life Technologies). The day before transfection, the cells were split to 0.5×10⁶/ml and cultured over night. To transfect the cells, 1×10⁶ cells were centrifuged at 90×G for 10 minutes and thereafter resuspended in 100 μl nucleofector solution (Amaxa® Cell Line Nucleofector® Kit V, Lonza) where 2 μg DNA (hPD-1 in pcDNA3) was added. The mixture was then transferred to a nucleofector cuvette. Cuvettes were placed in nucleofector II machine and nucleofected with program X-005. After incubation at room temperature (around 18-22° C.) for 10 min, 500 ml media was added to the cuvette and transferred to a 12-well plate containing 1 ml media. To select for transfected cells, geneticin was added at 1 mg/ml 48 hours after transfection. 10-14 days later, positive cells were purified into low, mid and high PD-1 expressors by FACS sorting on a FACSAria II machine. Thereafter, transfected cells were maintained in media containing 1 mg/ml geneticin.

### Quantification of PD-1

The basic principle of quantification using this set of beads is based on the fact that phytoerythrin (PE) labels antibody at a 1:1 ratio. Therefore, by using beads with defined numbers of PE molecules to generate a standard curve, the number of molecules of antibody bound to a cell can be determined.

The Jurkat cells were stained with PE labeled anti-PD1 antibody (EH12.2H7, BioLegend) or isotype control in FACS buffer (PBS with 2% FCS) at 4° C. for 30 minutes followed by washing in FACS buffer. One tube of Quantibrite™ beads (PE phytoerythrin quantification kit, BD bioscience (Cat. No. 340495) was re-suspended in 500 μl PBS.

Thereafter, the FACs machine was set up in such a way that Quantibrite™ beads and Jurkat cells can be run on the same settings. The beads were run until 10000 events were collected and from that a standard curve was generated as described by ED Biosciences for the PE Phycoerythrin Fluorescence Quantitation Kit, i.e. by plotting Log molecules per bead (lot specific information in the kit) versus Log MFI (fluorescence intensity) for the 4 populations of Quantibrite™ beads delivered. This standard curve was then used to calculate the number of molecules on the cell line by converting their MFI to number of molecules. Given that the antibodies are used at saturating concentrations and there is a 1:1 binding of antibodies to PD-1 molecules per cell, the number of antibodies bound per cell corresponds to the number of PD-1 molecules present per cell.

After that, the PD1-PE stained Jurkat cells were run on the FACS and the Log mean fluorescence intensity (MFI) for the samples of interest was used to calculate the number of antibodies bound.

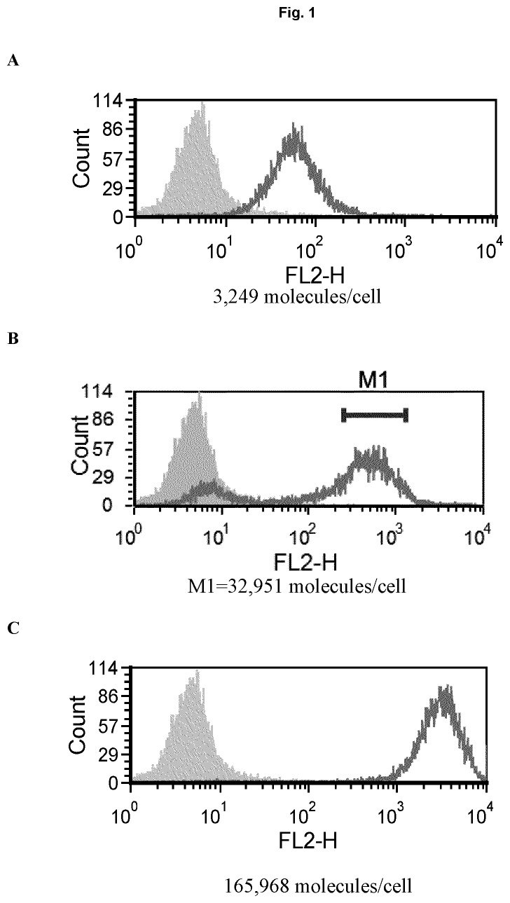

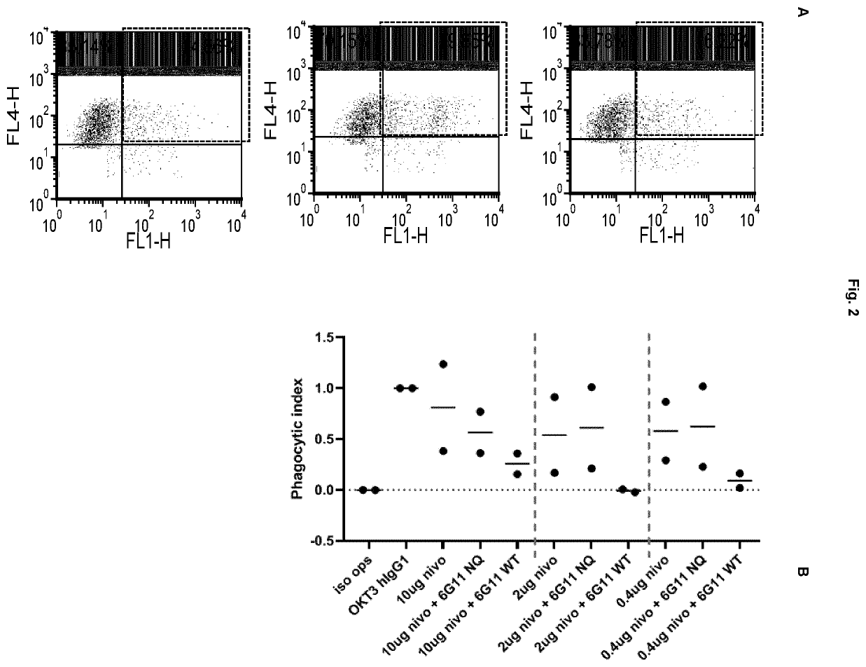

The results are shown in FIG. 1.

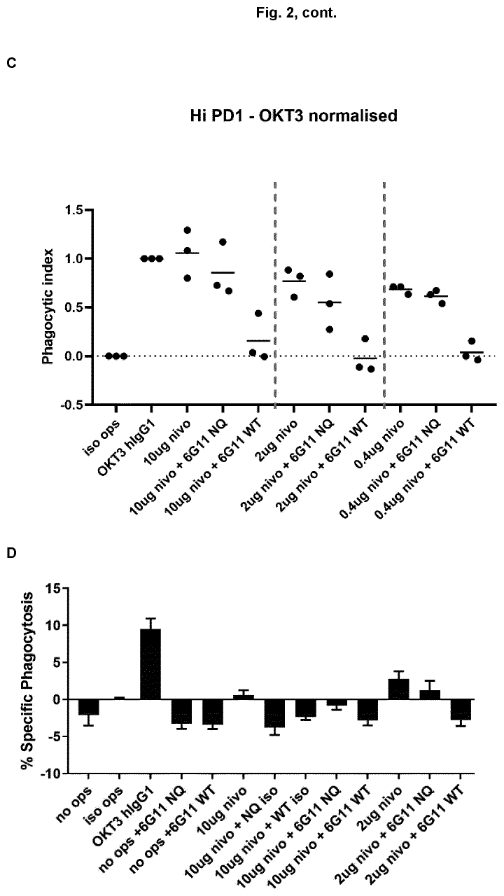

From FIG. 1 it is clear that the cell population having low expression comprises some cells (approximately 2%) having medium expression; however that is a neglectable part of the population, and it is clear from the phagocytosis experiment shown below (and demonstrated in FIG. 2) that this small part does not affect phagocytosis. Similarly, it is clear that the cell population having medium expression comprises some cells having low expression, but again this small fraction of cells does not affect the phagocytosis results, as shown below.

The low expressing subset (FIG. 1A) has an average of 3,249 PD-1 molecules/cell, with a bottom 5% cut off of 1,253 PD-1 molecules/cell and a top 5% cut off of 10,643 PD-1 molecules/cell. The medium expressing subset (FIG. 1B) has an average of 32,951 PD-1 molecules/cell, with a bottom 5% cut off of 15,498 PD-1 molecules/cell and a top 5% cut off of 77,822 PD-1 molecules/cell. The high expressing subset (FIG. 1C) has an average of 165,968 PD-1 molecules/cell, with a bottom 5% cut off of 65,406 PD-1 molecules/cell and a top 5% cut off of 390,946 PD-1 molecules/cell. The top and bottom 5% cut off values are provided for less overlap between the different subsets. The bottom 5% cut off value of 15,498, i.e. approximately 15,500, PD-1 molecules/cell as measured above, is used herein to define the lower limit of medium or high expression.

### Phagocytosis

Human PBMCs isolated from leukocyte cones obtained from the National Blood Service in Southampton was incubated in plates with RPMI medium (Life Technologies) containing glutamine, pyruvate, PenStrep and 1% heat inactivated human serum from Sigma Heat for 2 hours to allow monocytes to adhere. Media was then replaced with RPMI medium containing glutamine, pyruvate, PenStrep and +10% FCS (Sigma) After 24 hours MCSF (produced at the University of Southampton) was added. Macrophages were derived over 7 days with 2 media changes (including MCSF). Thereafter, macrophages were harvested by removing media, adding 2 ml PBS and placing on ice for 15 minutes before lightly scraping. The macrophages were then re-plated in 96 well plate for 2 hours. Macrophages were pre-treated with anti-hFcγRIIb mAbs (6G11 WT or 6G11 NQ) for 45 minutes at 2× final concentration before CFSE (Molecular probes) labelled Jurkats, opsonised with nivolumab (hlgG4) at 2× final concentration for 15 minutes, were added. The cells were co-culture for 1 hour at 37° C. before stained with anti-CD14 (BD-Bioscience), by incubating for 30 min in FACS-buffer at 4° C. followed by wash, and then read in a FACS machine.

The results are shown in FIG. 2.

### Example 2—PD-1 Quantification on Immune Cells from Tumor-Bearing Mice

PD-1 receptor numbers on immune cells from mice tumors were determined using Quantum™ Simply Cellular® beads (Bangs Laboratories, Inc.). In brief, beads were stained with rat anti PD-1 antibody (clone 29F.1A12, BioLegend) to create a standard curve. Cell samples were then read against the curve for determination of expression.

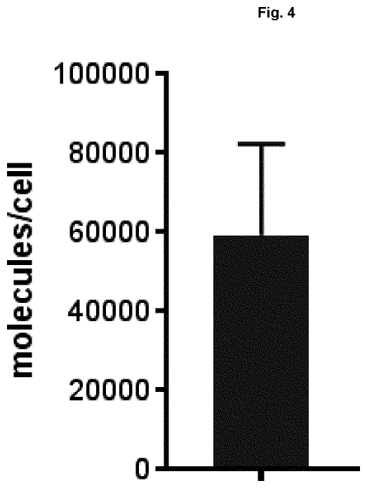

Quantification was done on cells from tumor-bearing mice. Mice were bred and maintained in local facilities in accordance with home office guidelines. Six to eight weeks-old female C57/BL6 mice were supplied by Taconic (Bomholt, Denmark) and maintained in local animal facilities. MC38 cells (ATCC) were grown in glutamax buffered RPMI supplemented with 10% FBS. When cells were semi confluent they were detached with trypsin and re-suspended in sterile PBS at 10×10⁶ cells/ml. Mice were s.c. injected with 100 μl cell suspension corresponding to 1×10⁶ cells/mouse. Tumors were grown for ˜20 days before collected. CD8+ T cell subsets were identified by FACS using CD45, CD3, CD4, and CD8 markers (all from BD Biosciences). PD-1 expression on different T cell subsets were quantified using a commercial rat anti PD-1 antibody (clone 29F.1A12) with corresponding isotype control (BioLegend). The results are shown in FIG. 4.

### Example 3—Combinational Effect with Anti PD-1 In-Vivo

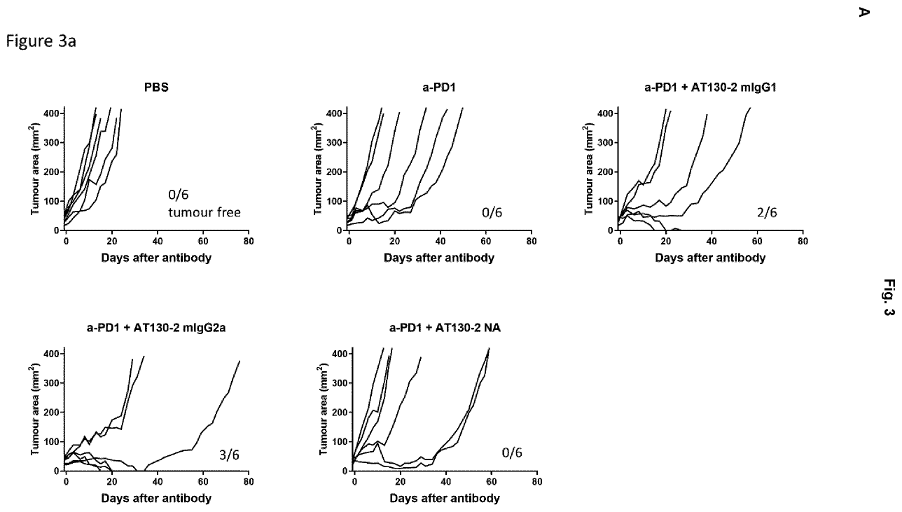

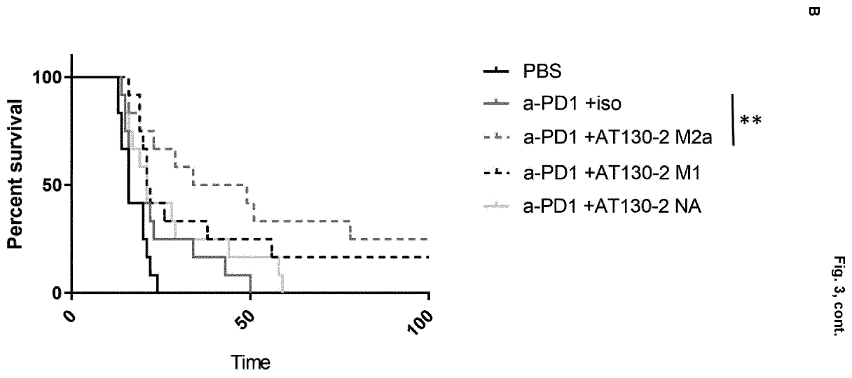

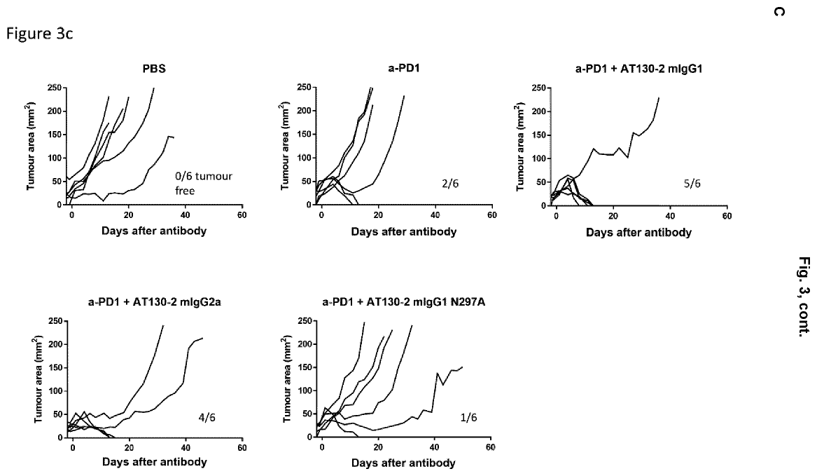

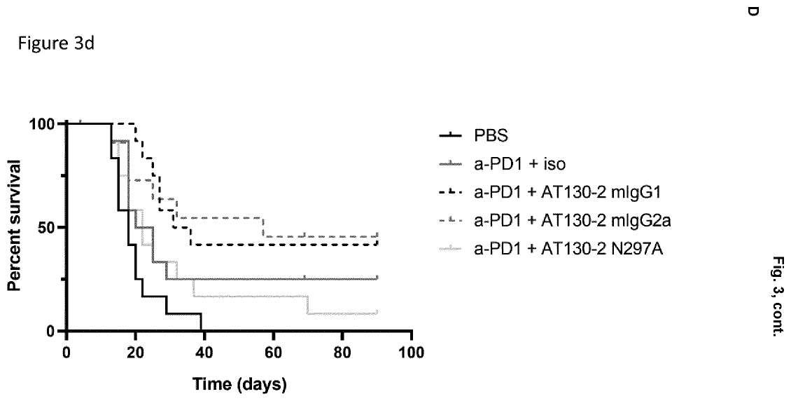

The PD-1 expression on mice cells correspond to expression levels between ‘mid and high’ on transfected Jurkat cells (Example 1, FIG. 1). In a phagocytosis assay, BI-1206 was shown to significantly reduce the level of phagocytosis for these ‘mid and high’ PD-1 expressing cells (Example 1, FIG. 2). This data, in combination with the improved therapeutic anti-tumor effect seen when combining anti PD-1 with anti-FcγRIIb (BI-1206 mouse surrogate) in the MC38 model in-vivo (FIG. 3), suggests an improved therapeutic effect of anti PD-1 in combination with BI-1206 in patients with a medium or high PD-1 expression, i.e. a PD-1 expression that is equal to or higher than 15,500 PD-1 molecules/cell.

### Example 4—Quantification of PD-1 Expression on Human T Cells

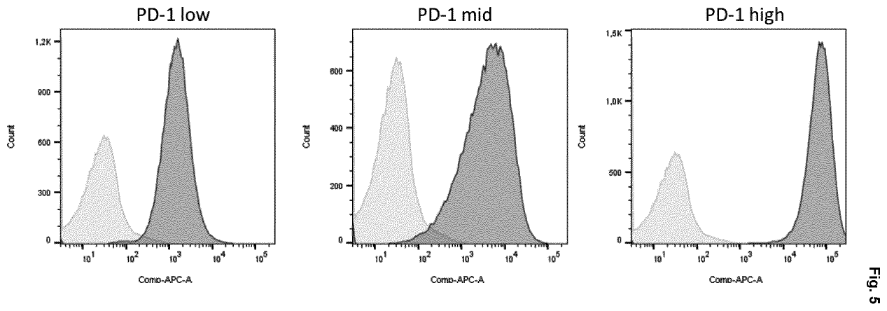

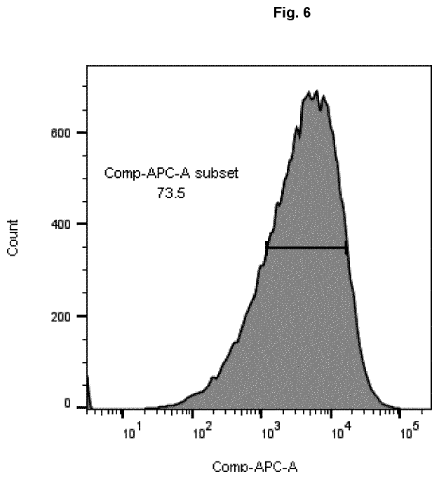

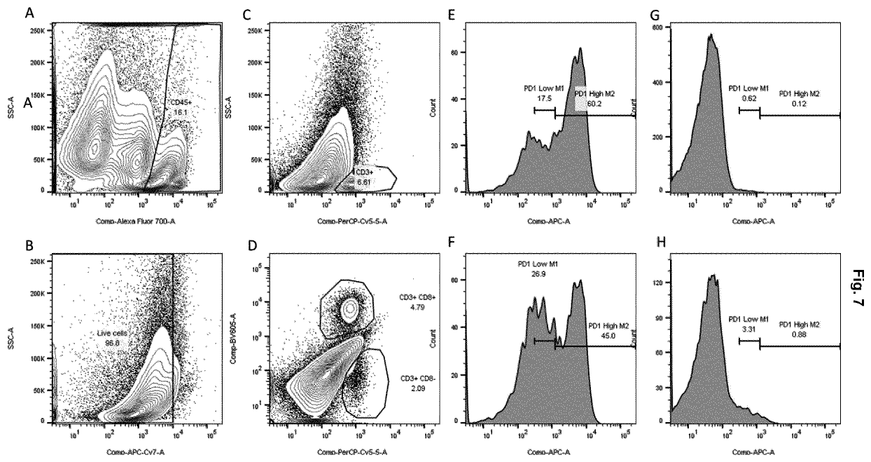

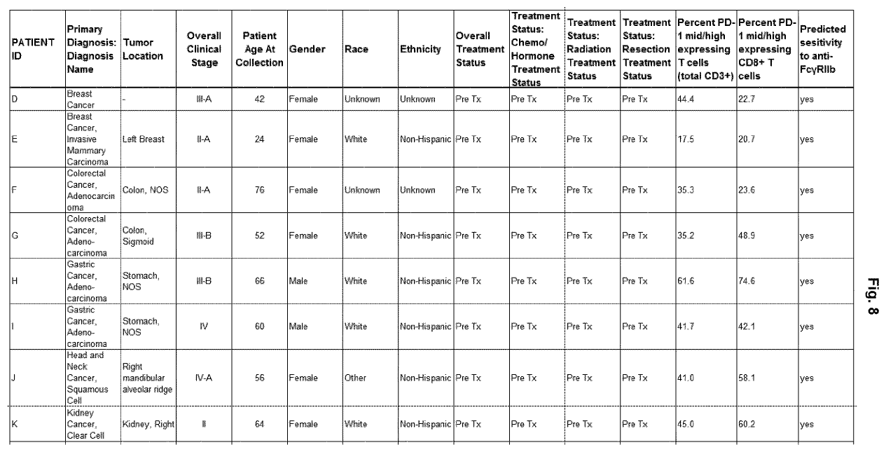

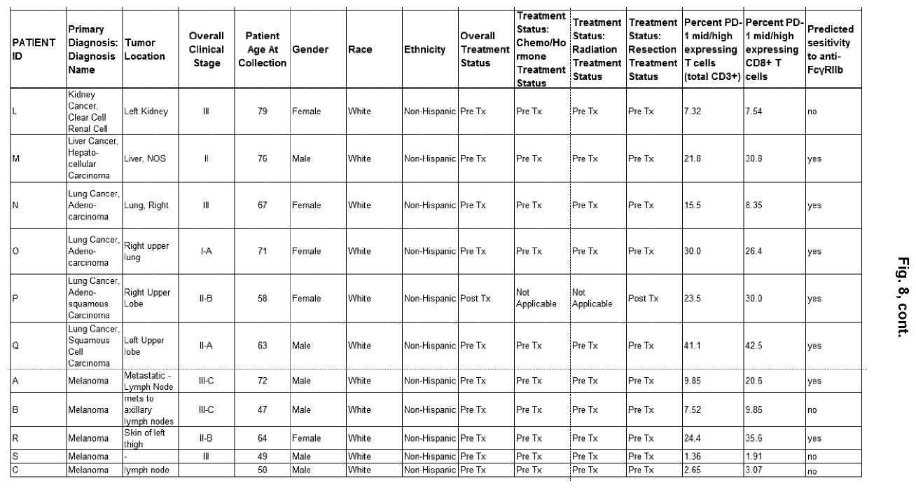

Dissociated and viable frozen tumor samples (see the Table in FIG. 8) were purchased from Discovery Life Sciences. The cells were thawed and washed in phosphate-buffered saline (PBS) prior to staining with a mix of the following antibodies: Alexa Fluor 700 mouse anti-human CD45 (clone H130, BD 560566), BV605 mouse anti-human CD8 (clone SK1, BD 564116), PerCP-Cy5.5 mouse anti-human CD3 (clone UCHT1, ED 560835), Alexa Fluor 647 human anti-human PD-1 (Pembrolizumab (KEYTRUDA), Clinical grade, Lot #8SNL80406, Merck Sharp & Dohme Limited). Fixable Viability Dye eFluor 780 was also included in the antibody staining mix (Invitrogen, 65-0865-14). Staining was performed in ED Horizon Brilliant Stain Buffer (BD 563794). The anti-human PD-1 was conjugated in-house with Alexa Fluor 647 and used at a receptor saturating concentration (5.5 μg/ml), shown by pre-titration experiments. The remaining antibodies were used at the concentrations recommended by the manufacturer. Cells were incubated with antibodies for 20 minutes and then washed and resuspended in PBS before acquisition using a BD FACSAria II. Analysis was done using the FlowJo software. The PD-1 expression analyses on PD-1 transfected Jurkat cells were done in a similar manner but only including Alexa Fluor 647 human anti-human PD-1 and Fixable Viability Dye eFluor 780 in the staining mix. The results are shown in FIG. 5. In tumor samples PD-1 expression was defined within the CD3+ and CD3+CD8+ population, respectively, pre-gated on live CD45+ cells (FIG. 7). PD-1 high gate were defined based on the PD-1 transfected Jurkat cells and were at the lower end set according to the low end of the full width/half height gate on the PD-1 mid Jurkat cells (FIG. 6).

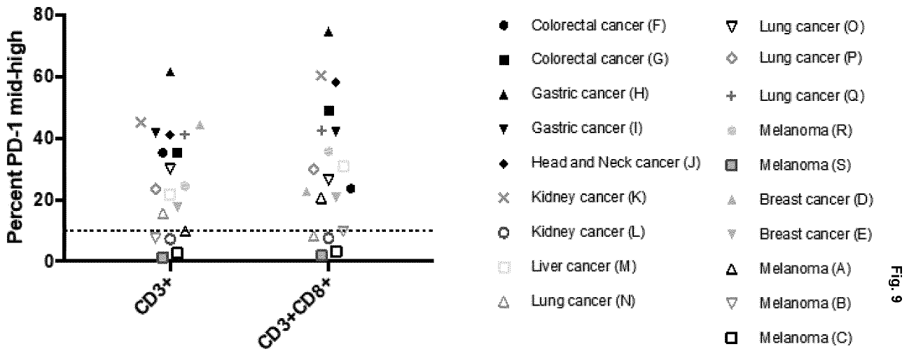

FIG. 9 shows percentage of PD-1 medium-high expressing CD3+ lymphocytes and CD3+CD8+ lymphocytes, respectively, in the individual tumor samples obtained from different patients. Patients with 10% of T cells expressing PD-1 at medium or high level are expected to benefit from anti-FcγRIIb in combination with anti-PD-1, and therefore a dotted line at 10% expression has been included.

1. A combination of:
a first antibody molecule that specifically binds FcγRIIb via its Fab region, and that binds an Fcγ receptor via its Fc region, and
a second antibody molecule that specifically binds to PD-1 and that binds to at least one Fcγ receptor via its Fc region;
for use in the treatment of cancer in a patient having tumor infiltrating T lymphocytes with a medium or high PD-1 expression.

2. A pharmaceutical composition comprising:
(i) a first antibody molecule that specifically binds FcγRIIb via its Fab region, and that binds an Fcγ receptor via its Fc region, and
(ii) a second antibody molecule that specifically binds to PD-1 and that binds to at least one Fcγ receptor via its Fc region;
for use in the treatment of cancer in a patient having tumor infiltrating T lymphocytes with a medium or high PD-1 expression

3. A kit for use in the treatment of cancer in a patient having tumor infiltrating T lymphocytes with a medium or high PD-1 expression comprising:
(i) a first antibody molecule that specifically binds FcγRIIb via its Fab region, and that binds an Fcγ receptor via its Fc region, and
(ii) a second antibody molecule that specifically binds to PD-1 and that binds to at least one Fcγ receptor via its Fc region.

4. Use of:
(i) a first antibody molecule that specifically binds FcγRIIb via its Fab region, and that binds an Fcγ receptor via its Fc region, and
(ii) a second antibody molecule that specifically binds to PD-1 and that binds to at least one Fcγ receptor via its Fc region;
in the manufacture of a medicament for use in the treatment of cancer in a patient having tumor infiltrating T lymphocytes with a medium or high PD-1 expression.

5. A method for treatment of cancer in a patient having tumor infiltrating T lymphocytes with a medium or high PD-1 expression, comprising administering:
(i) a first antibody molecule that specifically binds FcγRIIb via its Fab region, and that binds an Fcγ receptor via its Fc region, and
(ii) a second antibody molecule that specifically binds to PD-1 and that binds to at least one Fcγ receptor via its Fc region.

6. A diagnostic test for determining if a patient will benefit from combined treatment with:
(i) a first antibody molecule that specifically binds FcγRIIb via its Fab region, and that binds an Fcγ receptor via its Fc region, and
(ii) a second antibody molecule that specifically binds to PD-1 and that binds to at least one Fcγ receptor via its Fc region,
which test comprises determining the PD-1 expression on the patient's tumor infiltrating T lymphocytes, wherein medium or high PD-1 expression indicates that the patient will benefit from combined treatment.

7. A combination for use according to claim 1, a pharmaceutical composition for use according to claim 2, a kit according to claim 3, a use according to claim 4, a method according to claim 5 or a diagnostic test according to claim 6, wherein the patient's tumor infiltrating CD3 positive T lymphocytes have a medium or high PD-1 expression.

8. A combination for use, a pharmaceutical composition for use, a kit, a use, a method or a diagnostic test according to claim 7, wherein at least 10% of the patient's tumor infiltrating CD3 positive T lymphocytes have a medium or high PD-1 expression.

9. A combination for use, a pharmaceutical composition for use, a kit, a use, a method or a diagnostic test according to claim 7 or 8, wherein the patient's tumor infiltrating CD3 positive, CD8 positive T lymphocytes have a medium or high PD-1 expression.

10. A combination for use, a pharmaceutical composition for use, a kit, a use, a method or a diagnostic test according to claim 9, wherein at least 10% of the patient's tumor infiltrating CD3 positive, CD8 positive T lymphocytes have a medium or high PD-1 expression.

11. A combination for use, a pharmaceutical composition for use, a kit, a use, a method or a diagnostic test according to any one of the claims 1-10, wherein medium or high PD-1 expression is defined as at least 10% of the tumor infiltrating T lymphocytes in a sample from the patient having an expression of at least 15,500 PD-1 molecules per T lymphocyte.

12. A combination for use, a pharmaceutical composition for use, a kit, a use, a method or a diagnostic test according to claim 11, wherein medium or high PD-1 expression is measured using the anti-PD1 antibody EH12.2H7.

13. A combination for use, a pharmaceutical composition for use, a kit, a use, a method or a diagnostic test according to any one of the claims 1-12, wherein the cancer is solid cancer.

14. A combination for use, a pharmaceutical composition for use, a kit, a use, a method or a diagnostic test according to claim 13, wherein the solid cancer is selected from the group consisting of melanoma, lung cancer, head and neck cancer, Hodgkin lymphoma, primary mediastinal B-cell lymphoma (PMBCL), bladder cancer, colorectal cancer, gastric cancer, cervical cancer, liver cancer, Merkel cell carcinoma, kidney cancer and cutaneous squamous cell carcinoma.

15. A combination for use, a pharmaceutical composition for use, a kit, a use, a method or a diagnostic test according to claim 13 or 14, wherein the cancer is refractory.

16. A combination for use, a pharmaceutical composition for use, a kit, a use, a method or a diagnostic test according to any one of the claims 1-15, wherein the first antibody molecule and/or the second antibody molecule is selected from the group consisting of a human antibody molecule, a humanized antibody molecule, and an antibody molecule of human origin.

17. A combination for use, a pharmaceutical composition for use, a kit, a use, a method or a diagnostic test according to any one of the claims 1-16, wherein the first antibody molecule and/or the second antibody molecule is a monoclonal antibody molecule or an antibody molecule of monoclonal origin.

18. A combination for use, a pharmaceutical composition for use, a kit, a use, a method or a diagnostic test according to any one of the claims 1-17, wherein the first antibody molecule and/or the second antibody molecule is selected from the group consisting of a full-size antibody, a chimeric antibody, a single chain antibody, and an antigen-binding fragment thereof retaining the ability to bind an Fc receptor via its Fc region.

19. A combination for use, a pharmaceutical composition for use, a kit, a use, a method or a diagnostic test according to any one of the claims 1-18, wherein the first antibody molecule and/or the second antibody molecule is a human IgG antibody, a humanized IgG antibody molecule or an IgG antibody molecule of human origin.

20. A combination for use, a pharmaceutical composition for use, a kit, a use, a method or a diagnostic test according to claim 19, wherein the first antibody molecule is an IgG1 antibody molecule.

21. A combination for use, a pharmaceutical composition for use, a kit, a use, a method or a diagnostic test according to claim 19 or 20, wherein the second antibody molecule is an IgG4 antibody molecule.

22. A combination for use, a pharmaceutical composition for use, a kit, a use, a method or a diagnostic test according to any one of the claims 1-21, wherein the first antibody molecule and/or the second antibody molecule has been engineered for improved binding to activating Fc gamma receptors.

23. A combination for use, a pharmaceutical composition for use, a kit, a use, a method or a diagnostic test according to any one of the claims 1-22, wherein the first antibody molecule comprises a variable heavy chain (VH) comprising the following CDRs:
(i) SEQ ID NO: 51 and SEQ ID NO: 52 and SEQ ID NO: 53; or
(ii) SEQ ID NO: 57 and SEQ ID NO: 58 and SEQ ID NO: 59; or
(iii) SEQ ID NO: 63 and SEQ ID NO: 64 and SEQ ID NO: 65; or
(iv) SEQ ID NO: 69 and SEQ ID NO: 70 and SEQ ID NO: 71; or
(v) SEQ ID NO: 75 and SEQ ID NO: 76 and SEQ ID NO: 77; or
(vi) SEQ ID NO: 81 and SEQ ID NO: 82 and SEQ ID NO: 83; or
(vii) SEQ ID NO: 87 and SEQ ID NO: 88 and SEQ ID NO: 89; or
(viii) SEQ ID NO: 93 and SEQ ID NO: 94 and SEQ ID NO: 95; or
(ix) SEQ ID NO: 99 and SEQ ID NO: 100 and SEQ ID NO: 101; or
(x) SEQ ID NO: 105 and SEQ ID NO: 106 and SEQ ID NO: 107; or
(xi) SEQ ID NO: 111 and SEQ ID NO: 112 and SEQ ID NO: 113; or
(xii) SEQ ID NO: 117 and SEQ ID NO: 118 and SEQ ID NO: 119; or
(xiii) SEQ ID NO: 123 and SEQ ID NO: 124 and SEQ ID NO: 125; or
(xiv) SEQ ID NO: 129 and SEQ ID NO: 130 and SEQ ID NO: 131; or
(xv) SEQ ID NO: 135 and SEQ ID NO: 136 and SEQ ID NO: 137; or
(xvi) SEQ ID NO: 141 and SEQ ID NO: 142 and SEQ ID NO: 143; or
(xvii) SEQ ID NO: 147 and SEQ ID NO: 148 and SEQ ID NO: 149; or
(xviii) SEQ ID NO: 153 and SEQ ID NO: 154 and SEQ ID NO: 155; or
(xix) SEQ ID NO: 159 and SEQ ID NO: 160 and SEQ ID NO: 161; or
(xx) SEQ ID NO: 165 and SEQ ID NO: 166 and SEQ ID NO: 167; or
(xxi) SEQ ID NO: 171 and SEQ ID NO: 172 and SEQ ID NO: 173; or
(xxii) SEQ ID NO: 177 and SEQ ID NO: 178 and SEQ ID NO: 179; or
(xxiii) SEQ ID NO: 183 and SEQ ID NO: 184 and SEQ ID NO: 185; or
(xxiv) SEQ ID NO: 189 and SEQ ID NO: 190 and SEQ ID NO: 191.

24. A combination for use, a pharmaceutical composition for use, a kit, a use, a method or a diagnostic test according to any one of the claims 1-23, wherein the first antibody molecule comprises a variable light chain (VL) comprising the following CDRs:
(i) SEQ ID NO: 54 and SEQ ID NO: 55 and SEQ ID NO: 56; or
(ii) SEQ ID NO: 60 and SEQ ID NO: 61 and SEQ ID NO: 62; or
(iii) SEQ ID NO: 66 and SEQ ID NO: 67 and SEQ ID NO: 68; or
(iv) SEQ ID NO: 72 and SEQ ID NO: 73 and SEQ ID NO: 74; or
(v) SEQ ID NO: 78 and SEQ ID NO: 79 and SEQ ID NO: 80; or
(vi) SEQ ID NO: 84 and SEQ ID NO: 85 and SEQ ID NO: 86; or
(vii) SEQ ID NO: 90 and SEQ ID NO: 91 and SEQ ID NO: 92; or
(viii) SEQ ID NO: 96 and SEQ ID NO: 97 and SEQ ID NO: 98; or
(ix) SEQ ID NO: 102 and SEQ ID NO: 103 and SEQ ID NO: 104; or
(x) SEQ ID NO: 108 and SEQ ID NO: 109 and SEQ ID NO: 110; or
(xi) SEQ ID NO: 114 and SEQ ID NO: 115 and SEQ ID NO: 116; or
(xii) SEQ ID NO: 120 and SEQ ID NO: 121 and SEQ ID NO: 122; or
(xiii) SEQ ID NO: 126 and SEQ ID NO: 127 and SEQ ID NO: 128; or
(xiv) SEQ ID NO: 132 and SEQ ID NO: 133 and SEQ ID NO: 134; or
(xv) SEQ ID NO: 138 and SEQ ID NO: 139 and SEQ ID NO: 140; or
(xvi) SEQ ID NO: 144 and SEQ ID NO: 145 and SEQ ID NO: 146; or
(xvii) SEQ ID NO: 150 and SEQ ID NO: 151 and SEQ ID NO: 152; or
(xviii) SEQ ID NO: 156 and SEQ ID NO: 157 and SEQ ID NO: 158; or
(xix) SEQ ID NO: 162 and SEQ ID NO: 163 and SEQ ID NO: 164; or
(xx) SEQ ID NO: 168 and SEQ ID NO: 169 and SEQ ID NO: 170; or
(xxi) SEQ ID NO: 174 and SEQ ID NO: 175 and SEQ ID NO: 176; or
(xxii) SEQ ID NO: 180 and SEQ ID NO: 181 and SEQ ID NO: 182; or
(xxiii) SEQ ID NO: 186 and SEQ ID NO: 187 and SEQ ID NO: 188; or
(xxiv) SEQ ID NO: 192 and SEQ ID NO: 193 and SEQ ID NO: 194.

25. A combination for use, a pharmaceutical composition for use, a kit, a use, a method or a diagnostic test according to any one of the claims 1-24, wherein the first antibody molecule comprises a variable heavy chain (VH) amino acid sequence selected from the group consisting of: SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; and SEQ ID NO: 26.

26. A combination for use, a pharmaceutical composition for use, a kit, a use, a method or a diagnostic test according to any one of the claims 1-25, wherein the first antibody molecule comprises a variable light chain (VL) amino acid sequence selected from the group consisting of: SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 38; SEQ ID NO: 39; SEQ ID NO: 40; SEQ ID NO: 41; SEQ ID NO: 42; SEQ ID NO: 43; SEQ ID NO: 44; SEQ ID NO: 45; SEQ ID NO: 46; SEQ ID NO: 47; SEQ ID NO: 48; SEQ ID NO: 49; and SEQ ID NO: 50.

27. A combination for use, a pharmaceutical composition for use, a kit, a use, a method or a diagnostic test according to any one of the claims 1-26, wherein the first antibody molecule comprises the following CDR amino acid sequences:
(i) SEQ ID NO: 51 and SEQ ID NO: 52 and SEQ ID NO: 53 and SEQ ID NO: 54 and SEQ ID NO: 55 and SEQ ID NO: 56; or
(ii) SEQ ID NO: 57 and SEQ ID NO: 58 and SEQ ID NO: 59 and SEQ ID NO: 60 and SEQ ID NO: 61 and SEQ ID NO: 62; or
(iii) SEQ ID NO: 63 and SEQ ID NO: 64 and SEQ ID NO: 65 and SEQ ID NO: 66 and SEQ ID NO: 67 and SEQ ID NO: 68; or
(iv) SEQ ID NO: 69 and SEQ ID NO: 70 and SEQ ID NO: 71 and SEQ ID NO: 72 and SEQ ID NO: 73 and SEQ ID NO: 74; or
(v) SEQ ID NO: 75 and SEQ ID NO: 76 and SEQ ID NO: 77 and SEQ ID NO: 78 and SEQ ID NO: 79 and SEQ ID NO: 80; or
(vi) SEQ ID NO: 81 and SEQ ID NO: 82 and SEQ ID NO: 83 and SEQ ID NO: 84 and SEQ ID NO: 85 and SEQ ID NO: 86; or
(vii) SEQ ID NO: 87 and SEQ ID NO: 88 and SEQ ID NO: 89 and SEQ ID NO: 90 and SEQ ID NO: 91 and SEQ ID NO: 92; or
(viii) SEQ ID NO: 93 and SEQ ID NO: 94 and SEQ ID NO: 95 and SEQ ID NO: 96 and SEQ ID NO: 97 and SEQ ID NO: 98; or
(ix) SEQ ID NO: 99 and SEQ ID NO: 100 and SEQ ID NO: 101 and SEQ ID NO: 102 and SEQ ID NO: 103 and SEQ ID NO: 104; or
(x) SEQ ID NO: 105 and SEQ ID NO: 106 and SEQ ID NO: 107 and SEQ ID NO: 108 and SEQ ID NO: 109 and SEQ ID NO: 110; or
(xi) SEQ ID NO: 111 and SEQ ID NO: 112 and SEQ ID NO: 113 and SEQ ID NO: 114 and SEQ ID NO: 115 and SEQ ID NO: 116; or
(xii) SEQ ID NO: 117 and SEQ ID NO: 118 and SEQ ID NO: 119 and SEQ ID NO: 120 and SEQ ID NO: 121 and SEQ ID NO: 122; or
(xiii) SEQ ID NO: 123 and SEQ ID NO: 124 and SEQ ID NO: 125 and SEQ ID NO: 126 and SEQ ID NO: 127 and SEQ ID NO: 128; or
(xiv) SEQ ID NO: 129 and SEQ ID NO: 130 and SEQ ID NO: 131 and SEQ ID NO: 132 and SEQ ID NO: 133 and SEQ ID NO: 134; or
(xv) SEQ ID NO: 135 and SEQ ID NO: 136 and SEQ ID NO: 137 and SEQ ID NO: 138 and SEQ ID NO: 139 and SEQ ID NO: 140; or
(xvi) SEQ ID NO: 141 and SEQ ID NO: 142 and SEQ ID NO: 143 and SEQ ID NO: 144 and SEQ ID NO: 145 and SEQ ID NO: 146; or
(xvii) SEQ ID NO: 147 and SEQ ID NO: 148 and SEQ ID NO: 149 and SEQ ID NO: 150 and SEQ ID NO: 151 and SEQ ID NO: 152; or
(xviii) SEQ ID NO: 153 and SEQ ID NO: 154 and SEQ ID NO: 155 and SEQ ID NO: 156 and SEQ ID NO: 157 and SEQ ID NO: 158; or
(xix) SEQ ID NO: 159 and SEQ ID NO: 160 and SEQ ID NO: 161 and SEQ ID NO: 162 and SEQ ID NO: 163 and SEQ ID NO: 164; or
(xx) SEQ ID NO: 165 and SEQ ID NO: 166 and SEQ ID NO: 167 and SEQ ID NO: 168 and SEQ ID NO: 169 and SEQ ID NO: 170; or
(xxi) SEQ ID NO: 171 and SEQ ID NO: 172 and SEQ ID NO: 173 and SEQ ID NO: 174 and SEQ ID NO: 175 and SEQ ID NO: 176; or
(xxii) SEQ ID NO: 177 and SEQ ID NO: 178 and SEQ ID NO: 179 and SEQ ID NO: 180 and SEQ ID NO: 181 and SEQ ID NO: 182; or
(xxiii) SEQ ID NO: 183 and SEQ ID NO: 184 and SEQ ID NO: 185 and SEQ ID NO: 186 and SEQ ID NO: 187 and SEQ ID NO: 188; or
(xxiv) SEQ ID NO: 189 and SEQ ID NO: 190 and SEQ ID NO: 191 and SEQ ID NO: 192 and SEQ ID NO: 193 and SEQ ID NO: 194.

28. A combination for use, a pharmaceutical composition for use, a kit, a use, a method or a diagnostic test according to any one of the claims 1-27, wherein the first antibody molecule comprises the following amino acid sequences:
(i) SEQ ID NO: 3 and SEQ ID NO: 27; or
(ii) SEQ IS NO: 4 and SEQ ID NO: 28; or
(iii) SEQ IS NO: 5 and SEQ ID NO: 29; or
(iv) SEQ ID NO: 6 and SEQ ID NO: 30; or
(v) SEQ ID NO: 7 and SEQ ID NO: 31; or
(vi) SEQ ID NO: 8 and SEQ ID NO: 32; or
(vii) SEQ ID NO: 9 and SEQ ID NO: 33; or
(viii) SEQ ID NO: 10 and SEQ ID NO: 34; or
(ix) SEQ ID NO: 11 and SEQ ID NO: 35; or
(x) SEQ ID NO: 12 and SEQ ID NO: 36; or
(xi) SEQ ID NO: 13 and SEQ ID NO: 37; or
(xii) SEQ ID NO: 14 and SEQ ID NO: 38; or
(xiii) SEQ ID NO: 15 and SEQ ID NO: 39; or
(xiv) SEQ ID NO: 16 and SEQ ID NO: 40; or
(xv) SEQ ID NO: 17 and SEQ ID NO: 41; or
(xvi) SEQ ID NO: 18 and SEQ ID NO: 42; or
(xvii) SEQ ID NO: 19 and SEQ ID NO: 43; or
(xviii) SEQ ID NO: 20 and SEQ ID NO: 44; or
(xix) SEQ ID NO: 21 and SEQ ID NO: 45; or
(xx) SEQ ID NO: 22 and SEQ ID NO: 46; or
(xxi) SEQ ID NO: 23 and SEQ ID NO: 47; or
(xxii) SEQ ID NO: 24 and SEQ ID NO: 48; or
(xxiii) SEQ ID NO: 25 and SEQ ID NO: 49; or
(xxiv) SEQ ID NO: 26 and SEQ ID NO: 50.

29. A combination for use, a pharmaceutical composition for use, a kit, a use, a method or a diagnostic test according to any one of the claims 1-22, wherein the first antibody molecule is an antibody molecule that is capable of competing for binding to FcyRIIb with an antibody molecule as defined in any one of claims 23-28.


SEQUENCE LISTING (194 sequences)

SEQ ID NO: 1 - protein, 330 aa, Homo sapiens [sequence omitted]

SEQ ID NO: 2 - protein, 105 aa, Homo sapiens
  IgG constant region light (CL)
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 3 - protein, 118 aa, Artificial Sequence
  Antibody clone 1A01-VH variable region heavy (VH)
EVQLLESGGGLVQPGGSLRLSCAASGFTFSDYYMNWIRQTPGKGLEWVSLIGWDGGSTYYADSVKGRFTISRDNSENTLYLQMNSLRAEDTAVYYCARAYSGYELDYWGQGTLVTVSS

SEQ ID NO: 4 - protein, 117 aa, Artificial Sequence
  Antibody clone 1B07 variable region heavy (VH)
EVQLLESGGGLVQPGGSLRLSCAASGFTFSSYGMHWVRQAPGKGLEWVAFTRYDGSNKYYADSVRGRFTISRDNSKNTLYLQMNSLRAEDTAVYYCARENIDAFDVWGQGTLVTVSS

SEQ ID NO: 5 - protein, 122 aa, Artificial Sequence
  Antibody clone 1C04 variable region heavy (VH)
EVQLLESGGGLVQPGGSLRLSCAASGFTFSSYAMSWVRQAPGKGLEWVSSISDSGAGRYYADSVEGRFTISRDNSKNTLYLQMNSLRAEDTAVYYCARTHDSGELLDAFDIWGQGTLVTVSS

SEQ ID NO: 6 - protein, 124 aa, Artificial Sequence
  Antibody clone 1E05 variable region heavy (VH)
EVQLLESGGGLVQPGGSLRLSCAASGFTFSTYAMNWVRQVPGKGLEWVAVISYDGSNKNYVDSVKGRFTISRDNSKNTLYLQMNSLRAEDTAVYYCARNFDNSGYAIPDAFDIWGQGTLVTVSS

SEQ ID NO: 7 - protein, 121 aa, Artificial Sequence
  Antibody clone 2A09  variable region heavy (VH)
EVQLLESGGGLVQPGGSLRLSCAASGFTFSNAWMSWVRQAPGKGLEWVAYISRDADITHYPASVKGRFTISRDNSKNTLYLQMNSLRAEDTAVYYCTTGFDYAGDDAFDIWGQGTLVTVSS

SEQ ID NO: 8 - protein, 119 aa, Artificial Sequence
  Antibody clone 2B08 variable region heavy (VH)
EVQLLESGGGLVQPGGSLRLSCAASGFTFSDYYMSWVRQAPGKGLEWVALIGHDGNNKYYLDSLEGRFTISRDNSKNTLYLQMNSLRAEDTAVYYCARATDSGYDLLYWGQGTLVTVSS

SEQ ID NO: 9 - protein, 117 aa, Artificial Sequence
  Antibody clone 2E08 variable region heavy (VH)
EVQLLESGGGLVQPGGSLRLSCAASGFTFSDYYMSWIRQAPGKGLEWVSAIGFSDDNTYYADSVKGRFTISRDNSKNTLYLQMNSLRAEDTAVYYCAGGDGSGWSFWGQGTLVTVSS

SEQ ID NO: 10 - protein, 117 aa, Artificial Sequence
  Antibody clone 5C04 variable region heavy (VH)
EVQLLESGGGLVQPGGSLRLSCAASGFTFSNYGMHWVRQAPGKGLEWVAVISYDGSNKYYADSVKGRFTISRDNSKNTLYLQMNSLRAEDTAVYYCAREWRDAFDIWGQGTLVTVSS

SEQ ID NO: 11 - protein, 117 aa, Artificial Sequence
  Antibody clone 5C05 variable region heavy (VH)
EVQLLESGGGLVQPGGSLRLSCAASGFTFSTYGMHWVRQAPGKGLEWVAVISYDGSNKYYADSVKGRFTISRDNSKNTLYLQMNSLRAEDTAVYYCARENFDAFDVWGQGTLVTVSS

SEQ ID NO: 12 - protein, 117 aa, Artificial Sequence
  Antibody clone 5D07 variable region heavy (VH)
EVQLLESGGGLVQPGGSLRLSCAASGFTFSTYGMHWVRQAPGKGLEWVAVIAYDGSKKDYADSVKGRFTISRDNSKNTLYLQMNSLRAEDTAVYYCAREYRDAFDIWGQGTLVTVSS

SEQ ID NO: 13 - protein, 117 aa, Artificial Sequence
  Antibody clone 5E12 variable region heavy (VH)
EVQLLESGGGLVQPGGSLRLSCAASGFTFSSYGMHWVRQAPGKGLEWVAVISYDGINKDYADSMKGRFTISRDNSKNTLYLQMNSLRAEDTAVYYCARERKDAFDIWGQGTLVTVSS

SEQ ID NO: 14 - protein, 117 aa, Artificial Sequence
  Antibody clone 5G08 variable region heavy (VH)
EVQLLESGGGLVQPGGSLRLSCAASGFTFNNYGMHWVRQAPGKGLEWVAVISYDGSNRYYADSVKGRFTMSRDNSKNTLYLQMNSLRAEDTAVYYCARDRWNGMDVWGQGTLVTVSS

SEQ ID NO: 15 - protein, 119 aa, Artificial Sequence
  Antibody clone 5H06 variable region heavy (VH)
EVQLLESGGGLVQPGGSLRLSCAASGFTFSSYGMHWVRQAPGKGLEWVAVISYDGSDTAYADSVKGRFTISRDNSKNTLYLQMNSLRAEDTAVYYCARDHSVIGAFDIWGQGTLVTVSS

SEQ ID NO: 16 - protein, 119 aa, Artificial Sequence
  Antibody clone 6A09 variable region heavy (VH)
EVQLLESGGGLVQPGGSLRLSCAASGFTFSSYGMHWVRQAPGKGLEWVAVTSYDGNTKYYANSVKGRFTISRDNSKNTLYLQMNSLRAEDTAVYYCAREDCGGDCFDYWGQGTLVTVSS

SEQ ID NO: 17 - protein, 118 aa, Artificial Sequence
  Antibody clone 6B01 variable region heavy (VH)
EVQLLESGGGLVQPGGSLRLSCAASGFTFSNYGMHWVRQAPGKGLEWVAVISYDGSNKYYADSVKGRFTISRDNSKNTLYLQMNSLRAEDTAVYYCARDQLGEAFDIWGQGTLVTVSS

SEQ ID NO: 18 - protein, 117 aa, Artificial Sequence
  Antibody clone 6C11 variable region heavy (VH)
EVQLLESGGGLVQPGGSLRLSCAASGFTFDDYGMSWVRQAPGKGLEWVSAISGSGSSTYYADSVKGRFTISRDNSKNTLYLQMNSLRAEDTAVYYCAGGDIDYFDYWGQGTLVTVSS

SEQ ID NO: 19 - protein, 117 aa, Artificial Sequence
  Antibody clone 6C12 variable region heavy (VH)
EVQLLESGGGLVQPGGSLRLSCAASGFTFSSYGMHWVRQAPGKGLEWVAVISYDGSNKYYADSVKGRFTISRDNSKNTLYLQMNSLRAEDTAVYYCARERRDAFDIWGQGTLVTVSS

SEQ ID NO: 20 - protein, 119 aa, Artificial Sequence
  Antibody clone 6D01 variable region heavy (VH)
EVQLLESGGGLVQPGGSLRLSCAASGFTFSSYGMHWVRQAPGKGLEWVAVISYDGSNKYYADSVKGRFTISRDNSKNTLYLQMNSLRAEDTAMYYCARDHSAAGYFDYWGQGTLVTVSS

SEQ ID NO: 21 - protein, 119 aa, Artificial Sequence
  Antibody clone 6G03 variable region heavy (VH)
EVQLLESGGGLVQPGGSLRLSCAASGFTFGSYGMHWVRQAPGKGLEWVSGISWDSAIIDYAGSVKGRFTISRDNSKNTLYLQMNSLRAEDTAVYYCAKDEAAAGAFDIWGQGTLVTVSS

SEQ ID NO: 22 - protein, 121 aa, Artificial Sequence
  Antibody clone 6G08 variable region heavy (VH)
EVQLLESGGGLVQPGGSLRLSCAASGFTLSSYGISWVRQAPGKGLEWVSGISGSGGNTYYADSVKGRFTISRDNSKNTLYLQMNSLRAEDTAVYYCASSVGAYANDAFDIWGQGTLVTVSS

SEQ ID NO: 23 - protein, 117 aa, Artificial Sequence
  Antibody clone 6G11 variable region heavy (VH)
EVQLLESGGGLVQPGGSLRLSCAASGFTFSSYGMHWVRQAPGKGLEWMAVISYDGSNKYYADSVKGRFTISRDNSKNTLYLQMNSLRAEDTAVYYCARELYDAFDIWGQGTLVTVSS

SEQ ID NO: 24 - protein, 117 aa, Artificial Sequence
  Antibody clone 6H08 variable region heavy (VH)
EVQLLESGGGLVQPGGSLRLSCAASGFTFNNYGMHWVRQAPGKGLEWVAVISYDGSNKYYADSVKGRFTISKDNSKNTLYLQMNSLRAEDTAVYYCAREYKDAFDIWGQGTLVTVSS

SEQ ID NO: 25 - protein, 118 aa, Artificial Sequence
  Antibody clone 7C07 variable region heavy (VH)
EVQLLESGGGLVQPGGSLRLSCAASGFTFSSYGMHWVRQAPGKGLEWVAVISYDGSNKYYADSVKGRFTISRDNSQNTLYLQMNSLRAEDTAVYYCAREFGYIILDYWGQGTLVTVSS

SEQ ID NO: 26 - protein, 117 aa, Artificial Sequence
  Antibody clone 4B02 variable region heavy (VH)
EVQLLESGGGLVQPGGSLRLSCAASGFTFSNHGMHWVRQAPGKGLEWVAVISYDGTNKYYADSVRGRFTISRDNSKNTLYLQMNSLRAEDTAVYYCARETWDAFDVWGQGTLVTVSS

SEQ ID NO: 27 - protein, 111 aa, Artificial Sequence
  Antibody clone 1A01 variable region light (VL)
QSVLTQPPSASGTPGQRVTISCSGSSSNIGNNAVNWYQQLPGTAPKLLIYDNNNRPSGVPDRFSGSKSGTSASLAISGLRSEDEADYYCAAWDDSLNASIFGGGTKLTVLG

SEQ ID NO: 28 - protein, 111 aa, Artificial Sequence
  Antibody clone 1B07 variable region light (VL)
QSVLTQPPSASGTPGQRVTISCSGSSSNIGNNAVNWYQQLPGTAPKLLIYDNQQRPSGVPDRFSGSKSGTSASLAISGLRSEDEADYYCEAWDDRLFGPVFGGGTKLTVLG

SEQ ID NO: 29 - protein, 111 aa, Artificial Sequence
  Antibody clone 1C04 variable region light (VL)
QSVLTQPPSASGTPGQRVTISCSGSSSNIGSNHVLWYQQLPGTAPKLLIYGNSNRPSGVPDRFSGSKSGTSASLAISGLRSEDEADYYCAAWDDSLNGWVFGGGTKLTVLG

SEQ ID NO: 30 - protein, 112 aa, Artificial Sequence
  Antibody clone 1E05 variable region light (VL)
QSVLTQPPSASGTPGQRVTISCTGSSSNIGAGYDVHWYQQLPGTAPKLLIYDNNSRPSGVPDRFSGSKSGTSASLAISGLRSEDEADYYCAAWDDSLGGPVFGGGTKLTVLG

SEQ ID NO: 31 - protein, 112 aa, Artificial Sequence
  Antibody clone 2A09 variable region light (VL)
QSVLTQPPSASGTPGQRVTISCSGSSSNIGSNAVNWYQQLPGTAPKLLIYGNSDRPSGVPDRFSGSKSGTSASLAISGLRSEDEADYYCAAWDDSLNGRWVFGGGTKLTVLG

SEQ ID NO: 32 - protein, 111 aa, Artificial Sequence
  Antibody clone 2B08 variable region light (VL)
QSVLTQPPSASGTPGQRVTISCSGSSSNIGNNAVNWYQQLPGTAPKLLIYYDDLLPSGVPDRFSGSKSGTSASLAISGLRSEDEADYYCTTWDDSLSGVVFGGGTKLTVLG

SEQ ID NO: 33 - protein, 111 aa, Artificial Sequence
  Antibody clone 2E08 variable region light (VL)
QSVLTQPPSASGTPGQRVTISCSGSSSNIGNNAVNWYQQLPGTAPKLLIYDNNKRPSGVPDRFSGSKSGTSASLAISGLRSEDEADYYCATWDDSLRGWVFGGGTKLTVLG

SEQ ID NO: 34 - protein, 113 aa, Artificial Sequence
  Antibody clone 5C04 variable region light (VL)
QSVLTQPPSASGTPGQRVTISCTGSSSNIGAGYDVHWYQQLPGTAPKLLIYSDNQRPSGVPDRFSGSKSGTSASLAISGLRSEDEADYYCAAWDDSLSGSWVFGGGTKLTVLG

SEQ ID NO: 35 - protein, 113 aa, Artificial Sequence
  Antibody clone 5C05 variable region light (VL)
QSVLTQPPSASGTPGQRVTISCTGSSSNIGAGYDVHWYQQLPGTAPKLLIYSNSQRPSGVPDRFSGSKSGTSASLAISGLRSEDEADYYCAAWDDSLNGQVVFGGGTKLTVLG

SEQ ID NO: 36 - protein, 112 aa, Artificial Sequence
  Antibody clone 5D07 variable region light (VL)
QSVLTQPPSASGTPGQRVTISCTGSSSNIGAGYDVHWYQQLPGTAPKLLIYGNSNRPSGVPDRFSGSKSGTTASLAISGLRSEDEADYYCAAWDDSVSGWMFGGGTKLTVLG

SEQ ID NO: 37 - protein, 112 aa, Artificial Sequence
  Antibody clone 5E12 variable region light (VL)
QSVLTQPPSASGTPGQRVTISCTGSSSNIGAGYDVHWYQQLPGTAPKLLIYSNNQRPSGVPDRFSGSKSGTSASLAISGLRSEDEADYYCATWDDSLNGLVFGGGTKLTVLG

SEQ ID NO: 38 - protein, 113 aa, Artificial Sequence
  Antibody clone 5G08 variable region light (VL)
QSVLTQPPSASGTPGQRVTISCSGSSSNIGAGYDVHWYQQLPGTAPKLLIYANNQRPSGVPDRFSGSKSGTSASLAISGLRSEDEADYYCAAWDDSLNGPWVFGGGTKLTVLG

SEQ ID NO: 39 - protein, 110 aa, Artificial Sequence
  Antibody clone 5H06 variable region light (VL)
QSVLTQPPSASGTPGQRVTISCSGSSSNIGSNTVNWYQQLPGTAPKLLIYDNNKRPSGVPDRFSGSKSGTSASLAISGLRSEDEADYYCSSYAGSNNVVFGGGTKLTVLG

SEQ ID NO: 40 - protein, 112 aa, Artificial Sequence
  Antibody clone 6A09 variable region light (VL)
QSVLTQPPSASGTPGQRVTISCTGSSSNIGAGYDVHWYQQLPGTAPKLLIYGNSNRPSGVPDRFSGSKSGTSASLAISGLRSEDEADYYCAAWDDSLNEGVFGGGTKLTVLG

SEQ ID NO: 41 - protein, 112 aa, Artificial Sequence
  Antibody clone 6B01 variable region light (VL)
QSVLTQPPSASGTPGQRVTISCTGSSSNIGAGYDVHWYQQLPGTAPKLLIYDNNKRPSGVPDRFSGSKSGTSASLAISGLRSEDEADYYCATWDDSLSGPVFGGGTKLTVLG

SEQ ID NO: 42 - protein, 112 aa, Artificial Sequence
  Antibody clone 6C11 variable region light (VL)
QSVLTQPPSASGTPGQRVTISCTGSSSNFGAGYDVHWYQQLPGTAPKLLIYENNKRPSGVPDRFSGSKSGTSASLAISGLRSEDEADYYCAAWDDSLNGPVFGGGTKLTVLG

SEQ ID NO: 43 - protein, 110 aa, Artificial Sequence
  Antibody clone 6C12 variable region light (VL)
QSVLTQPPSASGTPGQRVTISCTGSSSNIGAGYDVHWYQQLPGTAPKLLIYSDNQRPSGVPDRFSGSKSGTSASLAISGLRSEDEADYYCATWDSDTPVFGGGTKLTVLG

SEQ ID NO: 44 - protein, 111 aa, Artificial Sequence
  Antibody clone 6D01 variable region light (VL)
QSVLTQPPSASGTPGQRVTISCSGSSSNIGSNTVNWYQQLPGTAPKLLIYGNSIRPSGGPDRFSGSKSGTSASLAISGLRSEDEADYYCASWDDSLSSPVFGGGTKLTVLG

SEQ ID NO: 45 - protein, 113 aa, Artificial Sequence
  Antibody clone 6G03 variable region light (VL)
QSVLTQPPSASGTPGQRVTISCTGSSSNIGAGYDVHWYQQLPGTAPKLLIYGNTDRPSGVPDRFSGSKSGTSASLAISGLRSEDEADYYCAAWDDSLSGPVVFGGGTKLTVLG

SEQ ID NO: 46 - protein, 112 aa, Artificial Sequence
  Antibody clone 6G08 variable region light (VL)
QSVLTQPPSASGTPGQRVTISCTGSSSNIGAGYDVHWYQQLPGTAPKLLIYGDTNRPSGVPDRFSGSKSGTSASLAISGLRSEDEADYYCAAWDDSLNGPVFGGGTKLTVLG

SEQ ID NO: 47 - protein, 112 aa, Artificial Sequence
  Antibody clone 6G11 variable region light (VL)
QSVLTQPPSASGTPGQRVTISCTGSSSNIGAGYDVHWYQQLPGTAPKLLIYADDHRPSGVPDRFSGSKSGTSASLAISGLRSEDEADYYCASWDDSQRAVIFGGGTKLTVLG

SEQ ID NO: 48 - protein, 109 aa, Artificial Sequence
  Antibody clone 6H08 variable region light (VL)
QSVLTQPPSASGTPGQRVTISCTGSSSNIGSNTVNWYQQLPGTAPKLLIYDNNKRPSGVPDRFSGSKSGTSASLAISGLRSEDEADYYCQAWGTGIRVFGGGTKLTVLG

SEQ ID NO: 49 - protein, 111 aa, Artificial Sequence
  Antibody clone 7C07 variable region light (VL)
QSVLTQPPSASGTPGQRVTISCSGSSSNIGSNTVNWYQQLPGTAPKLLIYRDYERPSGVPDRFSGSKSGTSASLAISGLRSEDEADYYCMAWDDSLSGVVFGGGTKLTVLG

SEQ ID NO: 50 - protein, 109 aa, Artificial Sequence
  Antibody clone 4B02 variable region light (VL)
QSVLTQPPSASGTPGQRVTISCSGSSSNIGSNNANWYQQLPGTAPKLLIYDNNKRPSGVPDRFSGSKSGTSASLAISGLRSEDEADYYCQAWDSSTVVFGGGTKLTVLG

SEQ ID NO: 51 - protein, 5 aa, Artificial Sequence
  Antibody clone 1A01 CDRH1
DYYMN

SEQ ID NO: 52 - protein, 17 aa, Artificial Sequence
  Antibody clone 1A01 CDRH2
LIGWDGGSTYYADSVKG

SEQ ID NO: 53 - protein, 9 aa, Artificial Sequence
  Antibody clone 1A01 CDRH3
AYSGYELDY

SEQ ID NO: 54 - protein, 13 aa, Artificial Sequence
  Antibody clone 1A01 CDRL1
SGSSSNIGNNAVN

SEQ ID NO: 55 - protein, 7 aa, Artificial Sequence
  Antibody clone 1A01 CDRL2
DNNNRPS

SEQ ID NO: 56 - protein, 11 aa, Artificial Sequence
  Antibody clone 1A01 CDRL3
AAWDDSLNASI

SEQ ID NO: 57 - protein, 5 aa, Artificial Sequence
  Antibody clone 1B07 CDRH1
SYGMH

SEQ ID NO: 58 - protein, 17 aa, Artificial Sequence
  Antibody clone 1B07 CDRH2
FTRYDGSNKYYADSVRG

SEQ ID NO: 59 - protein, 8 aa, Artificial Sequence
  Antibody clone 1B07 CDRH3
ENIDAFDV

SEQ ID NO: 60 - protein, 13 aa, Artificial Sequence
  Antibody clone 1B07 CDRL1
SGSSSNIGNNAVN

SEQ ID NO: 61 - protein, 7 aa, Artificial Sequence
  Antibody clone 1B07 CDRL2
DNQQRPS

SEQ ID NO: 62 - protein, 9 aa, Artificial Sequence
  Antibody clone 1B07 CDRL3
WDDRLFGPV

SEQ ID NO: 63 - protein, 5 aa, Artificial Sequence
  Antibody clone 1C04 CDRH1
SYAMS

SEQ ID NO: 64 - protein, 17 aa, Artificial Sequence
  Antibody clone 1C04 CDRH2
SISDSGAGRYYADSVEG

SEQ ID NO: 65 - protein, 13 aa, Artificial Sequence
  Antibody clone 1C04 CDRH3
THDSGELLDAFDI

SEQ ID NO: 66 - protein, 13 aa, Artificial Sequence
  Antibody clone 1C04 CDRL1
SGSSSNIGSNHVL

SEQ ID NO: 67 - protein, 7 aa, Artificial Sequence
  Antibody clone 1C04 CDRL2
GNSNRPS

SEQ ID NO: 68 - protein, 11 aa, Artificial Sequence
  Antibody clone 1C04 CDRL3
AAWDDSLNGWV

SEQ ID NO: 69 - protein, 5 aa, Artificial Sequence
  Antibody clone 1E05 CDRH1
TYAMN

SEQ ID NO: 70 - protein, 17 aa, Artificial Sequence
  Antibody clone 1E05 CDRH2
VISYDGSNKNYVDSVKG

SEQ ID NO: 71 - protein, 15 aa, Artificial Sequence
  Antibody clone 1E05 CDRH3
NFDNSGYAIPDAFDI

SEQ ID NO: 72 - protein, 14 aa, Artificial Sequence
  Antibody clone 1E05 CDRL1
TGSSSNIGAGYDVH

SEQ ID NO: 73 - protein, 7 aa, Artificial Sequence
  Antibody clone 1E05 CDRL2
DNNSRPS

SEQ ID NO: 74 - protein, 11 aa, Artificial Sequence
  Antibody clone 1E05 CDRL3
AAWDDSLGGPV

SEQ ID NO: 75 - protein, 5 aa, Artificial Sequence
  Antibody clone 2A09 CDRH1
NAWMS

SEQ ID NO: 76 - protein, 17 aa, Artificial Sequence
  Antibody clone 2A09 CDRH2
YISRDADITHYPASVKG

SEQ ID NO: 77 - protein, 12 aa, Artificial Sequence
  Antibody clone 2A09 CDRH3
GFDYAGDDAFDI

SEQ ID NO: 78 - protein, 13 aa, Artificial Sequence
  Antibody clone 2A09 CDRL1
SGSSSNIGSNAVN

SEQ ID NO: 79 - protein, 7 aa, Artificial Sequence
  Antibody clone 2A09 CDRL2
GNSDRPS

SEQ ID NO: 80 - protein, 12 aa, Artificial Sequence
  Antibody clone 2A09 CDRL3
AAWDDSLNGRWV

SEQ ID NO: 81 - protein, 5 aa, Artificial Sequence
  Antibody clone 2B08 CDRH1
DYYMS

SEQ ID NO: 82 - protein, 17 aa, Artificial Sequence
  Antibody clone 2B08 CDRH2
LIGHDGNNKYYLDSLEG

SEQ ID NO: 83 - protein, 10 aa, Artificial Sequence
  Antibody clone 2B08 CDRH3
ATDSGYDLLY

SEQ ID NO: 84 - protein, 13 aa, Artificial Sequence
  Antibody clone 2B08 CDRL1
SGSSSNIGNNAVN

SEQ ID NO: 85 - protein, 7 aa, Artificial Sequence
  Antibody clone 2B08 CDRL2
YDDLLPS

SEQ ID NO: 86 - protein, 11 aa, Artificial Sequence
  Antibody clone 2B08 CDRL3
TTWDDSLSGVV

SEQ ID NO: 87 - protein, 5 aa, Artificial Sequence
  Antibody clone 2E08 CDRH1
DYYMS

SEQ ID NO: 88 - protein, 17 aa, Artificial Sequence
  Antibody clone 2E08 CDRH2
AIGFSDDNTYYADSVKG

SEQ ID NO: 89 - protein, 8 aa, Artificial Sequence
  Antibody clone 2E08 CDRH3
GDGSGWSF

SEQ ID NO: 90 - protein, 13 aa, Artificial Sequence
  Antibody clone 2E08 CDRL1
SGSSSNIGNNAVN

SEQ ID NO: 91 - protein, 7 aa, Artificial Sequence
  Antibody clone 2E08 CDRL2
DNNKRPS

SEQ ID NO: 92 - protein, 11 aa, Artificial Sequence
  Antibody clone 2E08 CDRL3
ATWDDSLRGWV

SEQ ID NO: 93 - protein, 5 aa, Artificial Sequence
  Antibody clone 5C04 CDRH1
NYGMH

SEQ ID NO: 94 - protein, 17 aa, Artificial Sequence
  Antibody clone 5C04 CDRH2
VISYDGSNKYYADSVKG

SEQ ID NO: 95 - protein, 7 aa, Artificial Sequence
  Antibody clone 5C04 CDRH2
WRDAFDI

SEQ ID NO: 96 - protein, 14 aa, Artificial Sequence
  Antibody clone 5C04 CDRL1
TGSSSNIGAGYDVH

SEQ ID NO: 97 - protein, 7 aa, Artificial Sequence
  Antibody clone 5C04 CDRL2
SDNQRPS

SEQ ID NO: 98 - protein, 12 aa, Artificial Sequence
  Antibody clone 5C04 CDRL3
AAWDDSLSGSWV

SEQ ID NO: 99 - protein, 5 aa, Artificial Sequence
  Antibody clone 5C05 CDRH1
TYGMH

SEQ ID NO: 100 - protein, 17 aa, Artificial Sequence
  Antibody clone 5C05 CDRH2
VISYDGSNKYYADSVKG

SEQ ID NO: 101 - protein, 8 aa, Artificial Sequence
  Antibody clone 5C05 CDRH3
ENFDAFDV

SEQ ID NO: 102 - protein, 14 aa, Artificial Sequence
  Antibody clone 5C05 CDRL1
TGSSSNIGAGYDVH

SEQ ID NO: 103 - protein, 7 aa, Artificial Sequence
  Antibody clone 5C05 CDRL2
SNSQRPS

SEQ ID NO: 104 - protein, 12 aa, Artificial Sequence
  Antibody clone 5C05 CDRL3
AAWDDSLNGQVV

SEQ ID NO: 105 - protein, 5 aa, Artificial Sequence
  Antibody clone 5D07 CDRH1
TYGMH

SEQ ID NO: 106 - protein, 17 aa, Artificial Sequence
  Antibody clone 5D07 CDRH2
VIAYDGSKKDYADSVKG

SEQ ID NO: 107 - protein, 8 aa, Artificial Sequence
  Antibody clone 5D07 CDRH3
EYRDAFDI

SEQ ID NO: 108 - protein, 14 aa, Artificial Sequence
  Antibody clone 5D07 CDRL1
TGSSSNIGAGYDVH

SEQ ID NO: 109 - protein, 7 aa, Artificial Sequence
  Antibody clone 5D07 CDRL2
GNSNRPS

SEQ ID NO: 110 - protein, 11 aa, Artificial Sequence
  Antibody clone 5D07 CDRL3
AAWDDSVSGWM

SEQ ID NO: 111 - protein, 5 aa, Artificial Sequence
  Antibody clone 5E12 CDRH1
SYGMH

SEQ ID NO: 112 - protein, 17 aa, Artificial Sequence
  Antibody clone 5E12 CDRH2
VISYDGINKDYADSMKG

SEQ ID NO: 113 - protein, 8 aa, Artificial Sequence
  Antibody clone 5E12 CDRH3
ERKDAFDI

SEQ ID NO: 114 - protein, 14 aa, Artificial Sequence
  Antibody clone 5E12 CDRL1
TGSSSNIGAGYDVH

SEQ ID NO: 115 - protein, 7 aa, Artificial Sequence
  Antibody clone 5E12 CDRL2
SNNQRPS

SEQ ID NO: 116 - protein, 11 aa, Artificial Sequence
  Antibody clone 5E12 CDRL3
ATWDDSLNGLV

SEQ ID NO: 117 - protein, 5 aa, Artificial Sequence
  Antibody clone 5G08 CDRH1
NYGMH

SEQ ID NO: 118 - protein, 17 aa, Artificial Sequence
  Antibody clone 5G08 CDRH2
VISYDGSNRYYADSVKG

SEQ ID NO: 119 - protein, 8 aa, Artificial Sequence
  Antibody clone 5G08 CDRH3
DRWNGMDV

SEQ ID NO: 120 - protein, 14 aa, Artificial Sequence
  Antibody clone 5G08 CDRL1
SGSSSNIGAGYDVH

SEQ ID NO: 121 - protein, 7 aa, Artificial Sequence
  Antibody clone 5G08 CDRL2
ANNQRPS

SEQ ID NO: 122 - protein, 12 aa, Artificial Sequence
  Antibody clone 5G08 CDRL3
AAWDDSLNGPWV

SEQ ID NO: 123 - protein, 5 aa, Artificial Sequence
  Antibody clone 5H06 CDRH1
SYGMH

SEQ ID NO: 124 - protein, 17 aa, Artificial Sequence
  Antibody clone 5H06 CDRH2
VISYDGSDTAYADSVKG

SEQ ID NO: 125 - protein, 10 aa, Artificial Sequence
  Antibody clone 5H06 CDRH3
DHSVIGAFDI

SEQ ID NO: 126 - protein, 13 aa, Artificial Sequence
  Antibody clone 5H06 CDRL1
SGSSSNIGSNTVN

SEQ ID NO: 127 - protein, 7 aa, Artificial Sequence
  Antibody clone 5H06 CDRL2
DNNKRPS

SEQ ID NO: 128 - protein, 10 aa, Artificial Sequence
  Antibody clone 5H06 CDRL3
SSYAGSNNVV

SEQ ID NO: 129 - protein, 5 aa, Artificial Sequence
  Antibody clone 6A09 CDRH1
SYGMH

SEQ ID NO: 130 - protein, 17 aa, Artificial Sequence
  Antibody clone 6A09 CDRH2
VTSYDGNTKYYANSVKG

SEQ ID NO: 131 - protein, 10 aa, Artificial Sequence
  Antibody clone 6A09 CDRH3
EDCGGDCFDY

SEQ ID NO: 132 - protein, 14 aa, Artificial Sequence
  Antibody clone 6A09 CDRL1
TGSSSNIGAGYDVH

SEQ ID NO: 133 - protein, 7 aa, Artificial Sequence
  Antibody clone 6A09 CDRL2
GNSNRPS

SEQ ID NO: 134 - protein, 11 aa, Artificial Sequence
  Antibody clone 6A09 CDRL3
AAWDDSLNEGV

SEQ ID NO: 135 - protein, 5 aa, Artificial Sequence
  Antibody clone 6B01 CDRH1
NYGMH

SEQ ID NO: 136 - protein, 17 aa, Artificial Sequence
  Antibody clone 6B01 CDRH2
VISYDGSNKYYADSVKG

SEQ ID NO: 137 - protein, 9 aa, Artificial Sequence
  Antibody clone 6B01 CDRH3
DQLGEAFDI

SEQ ID NO: 138 - protein, 14 aa, Artificial Sequence
  Antibody clone 6B01 CDRL1
TGSSSNIGAGYDVH

SEQ ID NO: 139 - protein, 7 aa, Artificial Sequence
  Antibody clone 6B01 CDRL2
DNNKRPS

SEQ ID NO: 140 - protein, 11 aa, Artificial Sequence
  Antibody clone 6B01 CDRL3
ATWDDSLSGPV

SEQ ID NO: 141 - protein, 5 aa, Artificial Sequence
  Antibody clone 6C11 CDRH1
DYGMS

SEQ ID NO: 142 - protein, 17 aa, Artificial Sequence
  Antibody clone 6C11 CDRH2
AISGSGSSTYYADSVKG

SEQ ID NO: 143 - protein, 8 aa, Artificial Sequence
  Antibody clone 6C11 CDRH3
GDIDYFDY

SEQ ID NO: 144 - protein, 14 aa, Artificial Sequence
  Antibody clone 6C11 CDRL1
TGSSSNFGAGYDVH

SEQ ID NO: 145 - protein, 7 aa, Artificial Sequence
  Antibody clone 6C11 CDRL3
ENNKRPS

SEQ ID NO: 146 - protein, 11 aa, Artificial Sequence
  Antibody clone 6C11 CDRL3
AAWDDSLNGPV

SEQ ID NO: 147 - protein, 5 aa, Artificial Sequence
  Antibody clone 6C12 CDRH1
SYGMH

SEQ ID NO: 148 - protein, 17 aa, Artificial Sequence
  Antibody clone 6C12 CDRH2
VISYDGSNKYYADSVKG

SEQ ID NO: 149 - protein, 8 aa, Artificial Sequence
  Antibody clone 6C12 CDRH3
ERRDAFDI

SEQ ID NO: 150 - protein, 14 aa, Artificial Sequence
  Antibody clone 6C12 CDRL1
TGSSSNIGAGYDVH

SEQ ID NO: 151 - protein, 7 aa, Artificial Sequence
  Antibody clone 6C12 CDRL2
SDNQRPS

SEQ ID NO: 152 - protein, 9 aa, Artificial Sequence
  Antibody clone 6C12 CDRL3
ATWDSDTPV

SEQ ID NO: 153 - protein, 5 aa, Artificial Sequence
  Antibody clone 6D01 CDRH1
SYGMH

SEQ ID NO: 154 - protein, 17 aa, Artificial Sequence
  Antibody clone 6D01 CDRH2
VISYDGSNKYYADSVKG

SEQ ID NO: 155 - protein, 10 aa, Artificial Sequence
  Antibody clone 6D01 CDRH3
DHSAAGYFDY

SEQ ID NO: 156 - protein, 13 aa, Artificial Sequence
  Antibody clone 6D01 CDRL1
SGSSSNIGSNTVN

SEQ ID NO: 157 - protein, 7 aa, Artificial Sequence
  Antibody clone 6D01 CDRL2
GNSIRPS

SEQ ID NO: 158 - protein, 11 aa, Artificial Sequence
  Antibody clone 6D01 CDRL3
ASWDDSLSSPV

SEQ ID NO: 159 - protein, 5 aa, Artificial Sequence
  Antibody clone 6G03 CDRH1
SYGMH

SEQ ID NO: 160 - protein, 17 aa, Artificial Sequence
  Antibody clone 6G03 CDRH2
GISWDSAIIDYAGSVKG

SEQ ID NO: 161 - protein, 10 aa, Artificial Sequence
  Antibody clone 6G03 CDRH3
DEAAAGAFDI

SEQ ID NO: 162 - protein, 14 aa, Artificial Sequence
  Antibody clone 6G03 CDRL1
TGSSSNIGAGYDVH

SEQ ID NO: 163 - protein, 7 aa, Artificial Sequence
  Antibody clone 6G03 CDRL2
GNTDRPS

SEQ ID NO: 164 - protein, 12 aa, Artificial Sequence
  Antibody clone 6G03 CDRL3
AAWDDSLSGPVV

SEQ ID NO: 165 - protein, 5 aa, Artificial Sequence
  Antibody clone 6G08 CDRH1
SYGIS

SEQ ID NO: 166 - protein, 17 aa, Artificial Sequence
  Antibody clone 6G08 CDRH2
GISGSGGNTYYADSVKG

SEQ ID NO: 167 - protein, 12 aa, Artificial Sequence
  Antibody clone 6G08 CDRH3
SVGAYANDAFDI

SEQ ID NO: 168 - protein, 14 aa, Artificial Sequence
  Antibody clone 6G08 CDRL1
TGSSSNIGAGYDVH

SEQ ID NO: 169 - protein, 7 aa, Artificial Sequence
  Antibody clone 6G08 CDRL2
GDTNRPS

SEQ ID NO: 170 - protein, 11 aa, Artificial Sequence
  Antibody clone 6G08 CDRL3
AAWDDSLNGPV

SEQ ID NO: 171 - protein, 5 aa, Artificial Sequence
  Antibody clone 6G11 CDRH1
SYGMH

SEQ ID NO: 172 - protein, 17 aa, Artificial Sequence
  Antibody clone 6G11 CDRH2
VISYDGSNKYYADSVKG

SEQ ID NO: 173 - protein, 8 aa, Artificial Sequence
  Antibody clone 6G11 CDRH3
ELYDAFDI

SEQ ID NO: 174 - protein, 14 aa, Artificial Sequence
  Antibody clone 6G11 CDRL1
TGSSSNIGAGYDVH

SEQ ID NO: 175 - protein, 7 aa, Artificial Sequence
  Antibody clone 6G11 CDRL2
ADDHRPS

SEQ ID NO: 176 - protein, 11 aa, Artificial Sequence
  Antibody clone 6G11 CDRL3
ASWDDSQRAVI

SEQ ID NO: 177 - protein, 5 aa, Artificial Sequence
  Antibody clone 6H08 CDRH1
NYGMH

SEQ ID NO: 178 - protein, 17 aa, Artificial Sequence
  Antibody clone 6H08 CDRH2
VISYDGSNKYYADSVKG

SEQ ID NO: 179 - protein, 8 aa, Artificial Sequence
  Antibody clone 6H08 CDRH3
EYKDAFDI

SEQ ID NO: 180 - protein, 13 aa, Artificial Sequence
  Antibody clone 6H08 CDRL1
TGSSSNIGSNTVN

SEQ ID NO: 181 - protein, 7 aa, Artificial Sequence
  Antibody clone 6H08 CDRL2
DNNKRPS

SEQ ID NO: 182 - protein, 9 aa, Artificial Sequence
  Antibody clone 6H08 CDRL3
QAWGTGIRV

SEQ ID NO: 183 - protein, 5 aa, Artificial Sequence
  Antibody clone 7C07 CDRH1
SYGMH

SEQ ID NO: 184 - protein, 17 aa, Artificial Sequence
  Antibody clone 7C07 CDRH2
VISYDGSNKYYADSVKG

SEQ ID NO: 185 - protein, 9 aa, Artificial Sequence
  Antibody clone 7C07 CDRH3
EFGYIILDY

SEQ ID NO: 186 - protein, 13 aa, Artificial Sequence
  Antibody clone 7C07 CDRL1
SGSSSNIGSNTVN

SEQ ID NO: 187 - protein, 7 aa, Artificial Sequence
  Antibody clone 7C07 CDRL2
RDYERPS

SEQ ID NO: 188 - protein, 11 aa, Artificial Sequence
  Antibody clone 7C07 CDRL1
MAWDDSLSGVV

SEQ ID NO: 189 - protein, 5 aa, Artificial Sequence
  Antibody clone 4B02 CDRH1
NHGMH

SEQ ID NO: 190 - protein, 17 aa, Artificial Sequence
  Antibody clone 4B02 CDRH2
VISYDGTNKYYADSVRG

SEQ ID NO: 191 - protein, 8 aa, Artificial Sequence
  Antibody clone 4B02 CDRH3
ETWDAFDV

SEQ ID NO: 192 - protein, 13 aa, Artificial Sequence
  Antibody clone 4B02 CDRL1
SGSSSNIGSNNAN

SEQ ID NO: 193 - protein, 7 aa, Artificial Sequence
  Antibody clone 4B02 CDRL2
DNNKRPS

SEQ ID NO: 194 - protein, 9 aa, Artificial Sequence
  Antibody clone 4B02 CDRL3
QAWDSSTVV